SEQUENCE LISTING (35 sequences)

SEQ ID NO: 1 - RNA, 19 nt, Artificial Sequence
  Description of Artificial Sequence Synthetic oligonucleotide
nnngggganncnuccccnn

SEQ ID NO: 2 - RNA, 19 nt, Artificial Sequence
  Description of Artificial Sequence Synthetic oligonucleotide
nnnuggganncnucuuunn

SEQ ID NO: 3 - RNA, 22 nt, Artificial Sequence
  Description of Artificial Sequence Synthetic oligonucleotide
cgacauggacgugcagggggau

SEQ ID NO: 4 - RNA, 38 nt, Artificial Sequence
  Description of Artificial Sequence Synthetic oligonucleotide
auccccugcnnnuggganncnucuuunnguccgugucg

SEQ ID NO: 5 - RNA, 23 nt, Artificial Sequence
  Description of Artificial Sequence Synthetic oligonucleotide
gaagugcuucgauuuuggggugu

SEQ ID NO: 6 - RNA, 11 nt, Artificial Sequence
  Description of Artificial Sequence Synthetic oligonucleotide
acaccccaaaa

SEQ ID NO: 7 - RNA, 12 nt, Artificial Sequence
  Description of Artificial Sequence Synthetic oligonucleotide
ucgaagcacuuc

SEQ ID NO: 8 - RNA, 11 nt, Artificial Sequence
  Description of Artificial Sequence Synthetic oligonucleotide
cgaagcacuuc

SEQ ID NO: 9 - RNA, 10 nt, Artificial Sequence
  Description of Artificial Sequence Synthetic oligonucleotide
acaccccaaa

SEQ ID NO: 10 - RNA, 14 nt, Artificial Sequence
  Description of Artificial Sequence Synthetic oligonucleotide
aaaggcucuuuuca

SEQ ID NO: 11 - RNA, 23 nt, Artificial Sequence
  Description of Artificial Sequence Synthetic oligonucleotide
gaagugcuucgauuuuggggugu

SEQ ID NO: 12 - RNA, 44 nt, Artificial Sequence
  Description of Artificial Sequence Synthetic oligonucleotide
acaccccaaaaaaagguucuuuucagaguuacucgaagcacuuc

SEQ ID NO: 13 - RNA, 21 nt, Artificial Sequence
  Description of Artificial Sequence Synthetic oligonucleotide
nncnnnnncagwghnnnnnnn

SEQ ID NO: 14 - RNA, 24 nt, Artificial Sequence
  Description of Artificial Sequence Synthetic oligonucleotide
nnnncnnnnncagwghnnnnnnnn

SEQ ID NO: 15 - RNA, 22 nt, Artificial Sequence
  Description of Artificial Sequence Synthetic oligonucleotide
nncnnnnncagwghnnnunnnn

SEQ ID NO: 16 - RNA, 52 nt, Artificial Sequence
  Description of Artificial Sequence Synthetic oligonucleotide
nnnnnnnauganrrnnnnnnnaarnnnnnnnnnnnnnnyybgannnnnnnnn

SEQ ID NO: 17 - RNA, 383 nt, Human immunodeficiency virus
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguugggucgcgaaaggccuugugguacugccugauagggugcuugcgagugccccgggaggucucguagaccgugcaccaugagcacgaauccuaaaccucaaagaaaaaccaaacguaac

SEQ ID NO: 18 - RNA, 75 nt, Artificial Sequence
  Description of Artificial Sequence Synthetic oligonucleotide
ccugugaggaacuacugucuucacgcagaaagcgucuagccauggcguuaguaugagugucgugcagccuccagg

SEQ ID NO: 19 - RNA, 68 nt, Artificial Sequence
  Description of Artificial Sequence Synthetic oligonucleotide
nnnnnaacuacugucuucacgcagaaagcgucuagccauggcguuaguaugagugucgugcagnnnnn

SEQ ID NO: 20 - RNA, 21 nt, Artificial Sequence
  Description of Artificial Sequence Synthetic oligonucleotide
mmmggyyyuhhuhadddyymm

SEQ ID NO: 21 - RNA, 22 nt, Artificial Sequence
  Description of Artificial Sequence Synthetic oligonucleotide
mmmmggyyyuuyunarrrccmm

SEQ ID NO: 22 - RNA, 22 nt, Artificial Sequence
  Description of Artificial Sequence Synthetic oligonucleotide
nmmmggyyyuhhuharrrccmm

SEQ ID NO: 23 - RNA, 22 nt, Artificial Sequence
  Description of Artificial Sequence Synthetic oligonucleotide
cugggacaggaggaggaggcag

SEQ ID NO: 24 - RNA, 46 nt, Artificial Sequence
  Description of Artificial Sequence Synthetic oligonucleotide
cugcccguuucuuccucauugaaaaggcucuuuucagagccacuca

SEQ ID NO: 25 - RNA, 42 nt, Artificial Sequence
  Description of Artificial Sequence Synthetic oligonucleotide
caaacaccauugucaaaaggcucuuuucagagccacacucca

SEQ ID NO: 26 - RNA, 22 nt, Artificial Sequence
  Description of Artificial Sequence Synthetic oligonucleotide
uggagugugacaaugguguuug

SEQ ID NO: 27 - RNA, 17 nt, Artificial Sequence
  Description of Artificial Sequence Synthetic oligonucleotide
gugggggagaggcuguc

SEQ ID NO: 28 - RNA, 30 nt, Artificial Sequence
  Description of Artificial Sequence Synthetic oligonucleotide
cauccaccgagacuuucucuggaaacgaca

SEQ ID NO: 29 - RNA, 35 nt, Artificial Sequence
  Description of Artificial Sequence Synthetic oligonucleotide
acccaccgagacuuuucucggaaacaauucugacg

SEQ ID NO: 30 - RNA, 44 nt, Artificial Sequence
  Description of Artificial Sequence Synthetic oligonucleotide
uucacccaccgagacuuuucucggaaacccaaaauucugacuac

SEQ ID NO: 31 - RNA, 41 nt, Artificial Sequence
  Description of Artificial Sequence Synthetic oligonucleotide
acucaccgagacuuuucucggaaaaguuacuccuucuuugc

SEQ ID NO: 32 - RNA, 40 nt, Artificial Sequence
  Description of Artificial Sequence Synthetic oligonucleotide
auucaccgagacuuuucucggaaaccuucgaaccuucguu

SEQ ID NO: 33 - RNA, 19 nt, Artificial Sequence
  Description of Artificial Sequence Synthetic oligonucleotide
gagggcggguggaggagga

SEQ ID NO: 34 - RNA, 37 nt, Artificial Sequence
  Description of Artificial Sequence Synthetic oligonucleotide
acucaccgagacuuuucucggaaacccaaccuguucu

SEQ ID NO: 35 - RNA, 21 nt, Artificial Sequence
  Description of Artificial Sequence Synthetic oligonucleotide
mmmggyyyuhhuharrryymm

(19) United States
(12) Patent Application Publication
(10) Pub. No.: US 2016/0369271 A1
(43) Pub. Date: Dec. 22, 2016

(54) METHODS OF USING STRUCTURALLY INTERACTING RNA

(71) Applicants: HOCUSLOCUS, LLC, Albany, NY (US); The Research Foundation of the State University of New York, Albany, NY (US)

(72) Inventors: Francis J. Doyle, II, Albany, NY (US); Zachary E. Wurz, Amsterdam, NY (US); Scott Tenenbaum, Selkirk, NY (US); Edward Eveleth, Albany, NY (US)

(21) Appl. No.: 15/022,748

(22) PCT Filed: Sep. 17, 2014
(86) PCT No.: PCT/US14/56153

§ 371 Date: Mar. 17, 2016

Related U.S. Application Data
(60) Provisional application No. 61/878,711, filed on Sep. 17, 2013.

(51) Int. Cl. C12N 15/113; C12Q 1/68

(52) U.S. Cl. CPC C12N 15/113; C12Q 1/6876; C12N 2310/113; C12N 2310/531; C12N 2310/141; C12Q 2600/178

(57) ABSTRACT

Disclosed are RNA constructs which function to bind and/or inhibit a non-coding RNA (e.g., a miRNA). Such RNA constructs include an optionally weakened stem-loop structure stabilized by binding to a non-coding RNA. The non-coding RNA preferentially binds to the RNA construct as compared to a natural target (e.g., a mRNA). In certain embodiments, the RNA construct inhibits the function of the non-coding RNA. Such RNA constructs also have three-way junction joining regions 3′ and 5′ of the stem-loop structure.

### CROSS-REFERENCE TO RELATED APPLICATIONS

This application claims priority to and the benefit of U.S. Provisional Patent Application No. 61/878,711, filed Sep. 17, 2013, the disclosure of which is incorporated herein by reference in its entirety.

### FIELD OF THE INVENTION

The invention relates generally to recombinant or synthetic nucleic acid constructs that are capable of interacting with non-coding RNAs, e.g., a microRNA (miRNA or miR), such that the interaction modulates the structure of the nucleic acid construct, leading to a higher affinity binding of the non-coding RNA to the nucleic acid construct than to a natural target.

### BACKGROUND

MicroRNAs (miRNAs) are an abundant class of short endogenous RNAs that act as post-transcriptional regulators of gene expression by base-pairing with their target mRNAs. The approximately 22 nucleotide (nt) mature miRNAs are processed sequentially from longer hairpin transcripts (primary miRNA or precursor miRNA) by the RNAse III ribonucleases Drosha (Lee et al. (2003) SCIENCE, 299:1540) and Dicer (Hutvagner et al. (2001) SCIENCE 293: 834-838, Ketting et al. (2001) GENES DEV. 15: 2654-2659.) miRNAs function by recruiting the RNA-induced silencing complex (RISC) to cleave an mRNA target. RISC is a multiprotein complex that incorporates one strand of a small interfering RNA (siRNA) or microRNA (miRNA), and uses it as a template for recognizing complementary mRNA. When it finds a complementary strand, it activates argonaute (a protein within RISC) and cleaves the mRNA.

Since the discovery of miRNA, the creation of effective miRNA inhibitors (referred to in this application as anti-miRs) has been essential, first to study and verify miRNA interactions and secondly as a therapeutic tool. But, creating an effective anti-miR has proven challenging. Although a perfectly complimentary strand of RNA the same length as the miRNA may seem like the simplistic and ideal anti-miR, such an approach has limitations. For example, a perfectly complementary sequence can induce the RISC complex, inducing cleavage of the anti-miR and limiting the utility of this approach. To overcome this, a number of strategies have been rationally designed and tested as shown in FIG. 1. Three of these strategies, (FIG. 1A) a perfect complement, (FIG. 1B) masking (i.e., introducing a duplicate miRNA that binds to a target mRNA and “masks” the endogenous miRNA from recognizing the mRNA target), and (FIG. 1C) a RISC-loaded short hairpin RNA (a shRNA that targets the miRNA for degradation), are non-functional or showed only a modest inhibitory effect, according to Bak, et al. (2013) RNA 19(2):280-293. A more effective strategy, shown in FIG. 1D, is to introduce a mismatch or bulge in the middle of the anti-miR. Another strategy to reduce cleavage by the RISC complex is to put a stem-loop structure at both ends of the complementary sequence, as shown in FIG. 1E. While adding only stem-loops did not considerably improve inhibition, combining the bulge and two stem-loops strategies, FIG. 1F, more effectively inhibited the miRNA.

In addition to sequences and surrounding structures, chemical modifications to the RNA backbone can also be made to increase binding and decrease the likelihood of cleavage. This strategy may be acceptable for molecular tool use, but may cause hepatotoxicity when used as a therapeutic. Anti-miR therapies, including those targeting miR-122 (a liver-specific miRNA thought to be involved in fatty acid metabolism) and miR-155 (overexpressed in glioblastoma) for Hepatitis C Virus infection and brain cancer respectively hold promise. However, at present they require further improvements to reduce hepatotoxicity to fully enable their development and FDA approval.

Many commercial anti-miR suppliers suggest using 10× as much anti-miR as miRNA mimic (an ectopically added miRNA) before any effect can be seen, even over a broad 12 to 72 hour window. In other words, if 1× of an miRNA mimic produces an observable effect (inhibition of the miRNA target), it typically will require 10× of an anti-miR to see an observable effect (inhibition of an miRNA). The need for such large quantities for efficacy and the expansive timeframe make it clear that current commercial anti-miRs are inadequate.

In summary, none of these rationally designed approaches to creating anti-miRs are sufficient.

### SUMMARY OF THE INVENTION

The methods described herein relate, in part, to a method of inhibiting a non-coding RNA (e.g., a miRNA) using a nucleotide construct, wherein the non-coding RNA is capable of preferentially binding to the RNA construct as compared to a natural target (e.g., an mRNA). In certain embodiments, the method comprises contacting a miRNA with an RNA construct that inhibits the miRNA (i.e., an anti-miRNA or anti-miR), wherein the miRNA binds preferentially to the anti-miRNA as compared to a natural target, e.g., a complementary messenger RNA (mRNA), thereby inhibiting the miRNA. In certain embodiments, the RNA constructs (1) bind with high affinity to a non-coding RNA, (2) do not induce the RISC complex, and/or (3) require little to no chemical modification. These features can provide advantages for both therapeutic and research applications.

In certain aspects, the methods described herein provide an RNA construct which is an RNA that hybridizes with all or a portion of the non-coding RNA and which is selected such that the hybridization stabilizes the interaction between the RNA construct and non-coding RNA. In some embodiments, hybridization of the non-coding RNA to the RNA construct may cause a conformational change in the RNA construct that acts as a trigger to activate, suppress, or modulate a cascade of biochemical events, leading to some prophylactic or therapeutic effect. For example, hybridization of the non-coding RNA to the RNA construct may stabilize a stem-loop structure in the RNA construct to enhance binding of a cellular protein. In certain embodiments, a cellular protein can bind to the RNA construct, e.g., to the stem-loop of the RNA construct, whereby the cellular protein is inhibited. In certain embodiments, inhibition of the cellular protein occurs because the cellular protein is sequestered by the RNA construct and therefore unable to function.

Thus, one aspect of the invention involves an RNA construct that includes a non-naturally occurring, continuous sequence of ribonucleotide bases. These bases define a stem-loop structure. The RNA construct has simulated, three way junction joining regions 3′ and 5′ of the stem-loop structure. In addition, the RNA construct has a first region 5′ of the 5′ joining region, including bases complementary to a 3′ region of the non-coding RNA, and a second region 3′ of the 3′ joining region, including bases complementary to a 5′ region of the non-coding RNA. The non-coding RNA binds preferentially to the RNA construct compared to a natural target.

In certain embodiments, the base sequence of the first and second regions are selected to hybridize with complementary bases on the non-coding RNA and are spaced apart by an intermediate region on the non-coding RNA defining another three way junction joining region.

In certain embodiments, the non-coding RNA is an miRNA, an siRNA, a piRNA, an snoRNA, or an lncRNA. In certain embodiments, the RNA construct comprises a modified nucleic acid base. In certain embodiments, the RNA construct further comprises a detectable marker, for example, a fluorescent moiety or biotin.

In certain embodiments, the method further comprises transfecting a DNA encoding the RNA construct into a cell.

In certain embodiments, binding of the non-coding RNA to the RNA construct is detectable on a non-denaturing gel. In certain embodiments, binding of the non-coding RNA to the RNA construct is detectable on a denaturing gel. In certain embodiments, binding of the non-coding RNA to the RNA construct is detectable on a 1 M, 3 M, 7 M and/or 8 M denaturing urea gel.

In certain embodiments, the non-coding RNA is an miRNA and the RNA construct inhibits the function of the miRNA as determined by (a) measuring the quantity of a downstream target of the miRNA in the presence of the RNA construct; (b) measuring the quantity of the downstream target of the miRNA in the absence of the RNA construct; and (c) determining that the quantity of the downstream target in the presence of the RNA construct is greater that the quantity of the downstream target the absence of the RNA construct. The downstream target can be, for example, an mRNA or a protein.

In certain embodiments, the reduction in free energy that occurs when an RNA construct binds a non-coding RNA is at least about 10 kCal, at least about 25 kCal, at least about 50 kCal, at least about 100 kCal, at least about 1,000 kCal. In certain embodiments, the reduction in free energy that occurs when a non-coding RNA binds an RNA construct is at least 1% more, at least 2% more, at least 5% more, at least 10% more, at least 25% more, at least 50% more at least 75% more, at least 95% more, at least 100% more, at least 200% more, at least 500% more than the reduction in free energy that occurs when a non-coding RNA binds its natural target.

In certain embodiments, the non-coding RNA is an miRNA, and at least 50% of the miRNA remains uncleaved by the RISC complex.

In certain embodiments, the RNA construct inhibits the non-coding RNA and in the presence of the non-coding RNA, the construct assumes a stem-loop conformation promoting association with a cellular protein, whereby association of the stem-loop with the cellular protein inhibits the cellular protein. In certain embodiments, the RNA construct inhibits the non-coding RNA by sequestering the non-coding RNA, and association of the stem-loop with the cellular protein inhibits the cellular protein by sequestering the cellular protein. In certain embodiments, inhibition of the non-coding RNA and cellular protein is lethal to the cell.

In certain embodiments, a joining region comprises no more than about 10 nucleotides. In certain embodiments, at least one three-way junction joining region comprises at least one unpaired nucleotide. In certain embodiments, each three-way junction joining region comprises at least one unpaired nucleotide. In certain embodiments, at least one three-way junction joining region comprises at least one unpaired nucleotide, at least 2 unpaired nucleotides, at least 3 unpaired nucleotides, at least 4 unpaired nucleotides, at least 5 unpaired nucleotides, at least 6 unpaired nucleotides, at least 7 unpaired nucleotides, at least 8 unpaired nucleotides, at least 9 unpaired nucleotides, or at least 10 unpaired nucleotides. In certain embodiments, each three-way junction joining region comprises at least one unpaired nucleotide, at least 2 unpaired nucleotides, at least 3 unpaired nucleotides, at least 4 unpaired nucleotides, at least 5 unpaired nucleotides, at least 6 unpaired nucleotides, at least 7 unpaired nucleotides, at least 8 unpaired nucleotides, at least 9 unpaired nucleotides, or at least 10 unpaired nucleotides.

In certain embodiments, at least one base in the stem of the stem-loop structure is mismatched with its potential binding partner so as to reduce the stability of the stem-loop conformation.

In certain embodiments, the non-coding RNA is characteristic of a pathogen. In certain embodiments, the pathogen is a virus or a single-celled microorganism. In certain embodiments, the non-coding RNA is expressed preferentially in a cell type of a multicellular organism. In certain embodiments, the cell type is an infected cell or a neoplastic cell, and the non-coding RNA is expressed by an organism infecting the cell or by the neoplastic cell.

In certain embodiments, the RNA construct comprises more than one said stem-loop structure.

### BRIEF DESCRIPTION OF THE FIGURES

The foregoing and other objects, features, and advantages of the present invention, as well as the invention itself, may be more fully understood from the following description of preferred embodiments, when read together with the accompanying drawings.

FIG. 1 depicts prior art anti-miR strategies. FIG. 1A shows a perfectly complementary anti-miR, FIG. 1B shows masking, FIG. 1C shows a RISC-loaded shRNA, FIG. 1D shows an anti-miR with a mismatch or bulge in the middle, FIG. 1E shows placement of a stem-loop structure at both ends of the complementary sequence, and FIG. 1F shows an anti-miR combining a bulge and stem-loops.

FIG. 2 shows titration of RNA construct/non-coding RNA complex on Tris-Borate-EDTA (TBE)-urea denaturing gel.

FIG. 3 shows naturally-occurring interactions between mRNA and miRNA. These interactions suggest that perfect complementarity between the flanks of an RNA construct and a non-coding RNA is unnecessary.

FIGS. 4A-C show the components and nomenclature of a three-way junction. FIG. 4D shows the range of relative sizes of joining regions that give rise to the different three-way junction family members shown in FIG. 5. J1 corresponds to the junction region that connects the two helices most closely approaching a coaxial conformation. (See, e.g., Lescoute et al. (2006) RNA 12:83-93.)

FIG. 5 shows examples of three-way junction family types. An RNA construct can form three basic conformations when bound to a non-coding RNA, corresponding to the three family types (A, B and C) of three-way junctions. Three-way junction conformation can affect the ability of RBPs to bind to a stem-loop.

FIGS. 6A-D show a mock example of an RNA construct using a stem-loop element. FIG. 6A shows a consensus structure (5′ NNNGGGGANNCNUCCCCNN-3′ (SEQ ID NO:1)) of an RBP binding site, and the gray area denotes a region where point mutations may be made to weaken stem-loop structure (asterisks show bases that are required for protein binding). The shaded boxes of FIG. 6B show point mutations made to the primary structure (sequence) at spots calculated to increase Minimum Free Energy (MFE) and reduce spontaneous formation of required secondary structure for RBP binding. The sequence of this RNA construct is (5′ NNNUGGGANNCNUCUUUNN-3′ (SEQ ID NO:2)). FIGS. 6C and D show a simulated three way junction (depicted generally in FIG. 6C), that will form when a non-coding RNA hybridizes to the RNA construct (SEQ ID NO:2) to stabilize the formation of the RBP binding site.

FIGS. 7A-C show an example of the RNA construct (SEQ ID NO:2) of FIGS. 6A-D (FIG. 7A) designed to hybridize to a HCMV miRNA-US4 non-coding RNA (5′-CGACAUGGACGUGCAGGGGGAU-3′ (SEQ ID NO:3) (FIG. 7B). FIG. 7C shows the RNA construct with flank sequences (5′-AUCCCCUGCNNNUGGGANNCNUCUUUNNGUCCGUGUCG-3′) (SEQ ID NO:4), hybridized to the non-coding RNA to form a three-way junction.

FIGS. 8A-C show that the interaction between flank regions of an RNA construct and non-coding RNA (hsa-miR-373, mirBase No. MIMAT0000726; 5′-GAAGUGCUUCGAUUUUGGGGUGU-3′ (SEQ ID NO:5)) can produce J3 regions of varying sizes. FIG. 8A shows a J3 region of 0 nucleotides produced by the interaction of the non-coding RNA with the 5′ flank region ACACCCCAAAA (SEQ ID NO:6) and the 3′ flank region UCGAAGCACUUC (SEQ ID NO:7); FIG. 8B shows a J3 region of 1 nucleotide produced by the interaction of the non-coding RNA with the 5′ flank region ACACCCCAAAA (SEQ ID NO:6) and the 3′ flank region CGAAGCACUUC (SEQ ID NO:8); and FIG. 8C shows a J3 region of 2 nucleotides produced by the interaction of the non-coding RNA with the 5′ flank region ACACCCCAAA (SEQ ID NO:9) and the 3′ flank region CGAAGCACUUC (SEQ ID NO:8).

FIG. 9 shows an example of a SELEX approach to designing an RNA construct. A library of sequences is constructed in which regions containing nucleotides critical for RBP binding (5′-AAAGGCUCUUUUCA-3′ (SEQ ID NO:10), in this example) and regions designed to hybridize to a non-coding RNA are held constant, while remaining regions are varied at random.

FIG. 10 shows a model of the proposed biological mechanism of action of the Histone Stem Loop (HSL) motif (and an RNA construct based on a HSL motif). The stem-loop binding protein (SLBP) bound to an RNA construct may act through one or more complexes to promote translation of an attached polypeptide coding region.

FIG. 11 shows an example of an RNA construct based on histone stem-loop Consensus Sequence I. Two GU pairings and one UG pairing are designed at the lower portion of the stem to weaken it, such that formation of the stem-loop structure is dependent on the association with a non-coding RNA (hsa-mIR-373 (SEQ ID NO:11) in this example). This RNA construct has the nucleotide sequence 5′-ACACCCCAAAAAAAGGUUCUUUUCAGAGUUACUCGAAGCACUUC-3′ (SEQ ID NO:12)).

FIG. 12A shows an RNA construct incorporating a weakened Iron Response Element (IRE) motif that does not effectively form in the absence of a non-coding RNA. FIG. 12B shows the reconstituted IRE motif hybridized to a non-coding RNA. FIG. 12C shows examples of IRE1 (5′-NNCNNNNNCAGWGHNNNNNNN-3′ (SEQ ID NO:13)), IRE2 (5′-NNNNCNNNNNCAGWGHNNNNNNNN-3′ (SEQ ID NO:14)), and IRE3 (5′-NNCNNNNNCAGWGHNNNUNNNN-3′ (SEQ ID NO:15)) consensus sequences (left to right, respectively).

FIG. 13 shows an example of a SECIS consensus sequence (5′-NNNNNNNAUGANRRNNNNNNNAARNNNNNNNNNNNNYYBGANNNNNNNN N-3′) (SEQ ID NO:16).

FIGS. 14A-B show a motif from the internal ribosome entry site (IRES) element which can be used as the RBP target in an RNA construct. FIG. 14A shows the general structure of an IRES element with domains II-IV depicted. FIG. 14B shows an IRES element from the Hepatitis C virus (SEQ ID NO:17). The internal start codon (AUG), located in domain IV, is shaded.

FIGS. 15A-B show an exemplary portion of an IRES element that can be used as the basis of an RNA construct. This portion is the region of the internal ribosome entry site (IRES) element that corresponds to domain II in the Hepatitis C virus. FIG. 15A shows the sequence of domain II (SEQ ID NO:18) from the IRES of Hepatitis C virus. FIG. 15B shows the base stem region (“N” nucleotides enclosed in a box) of domain II of the Hepatitis C virus that can be weakened by introduction of non-canonical base pairings (SEQ ID NO:19).

FIG. 16A-B show non-coding RNA stabilization of an RNA construct incorporating an IRES motif FIG. 16A shows an RNA construct incorporating a weakened IRES motif that does not effectively form in the absence of a non-coding RNA. FIG. 16B shows the reconstituted IRES motif hybridized to a non-coding RNA.

FIG. 17 depicts an RNA construct comprising several MS2-RNA tags in tandem at the 5′-end and a histone stem-loop at the 3′ end.

FIG. 18 is a bar graph depicting that miR-4298 was efficiently immunoprecipitated relative to four other miRNAs (A-D).

FIG. 19 depicts an RNA construct of the invention, anti-miR-122, bound to a non-coding RNA, miR122.

FIG. 20 depicts a Western blot and a bar graph showing the corresponding quantitation of protein, indicating that CAT-1 expression increased nearly 3× in the presence of the sxRNA miR-122 anti-miR as compared to untreated cells or those treated with a competitor (Qiagen) miR-122 anti-miR.

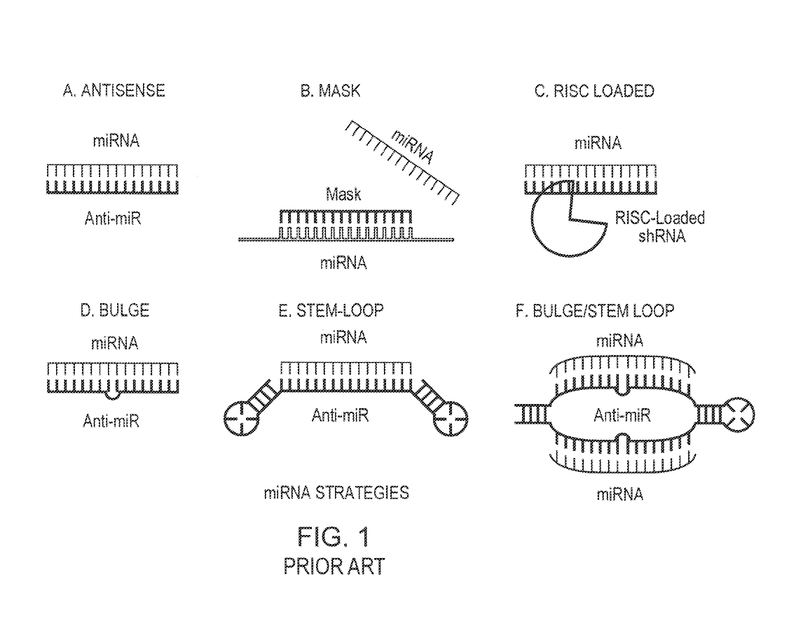

### DETAILED DESCRIPTION

### I. Introduction

The present invention relates to the use of interactions between a nucleic acid construct, such as an RNA construct, and a non-coding RNA. As disclosed herein, an RNA construct can be designed to bind to a non-coding RNA, wherein the non-coding RNA binds to the RNA construct preferentially as compared to a natural target. In certain embodiments, the stem-loop of the RNA construct binds to a cellular protein (e.g., eIF3; eIF4E, G, or A; aconitase 1; the 40S subunit) thereby inhibiting the cellular protein. In certain embodiments, the stem-loop of the RNA construct is stabilized by binding to a non-coding RNA. Stabilization of the RNA construct by the non-coding RNA can promote the interaction of the stem-loop structure with a cellular protein thereby inhibiting the cellular protein. In certain embodiments, the cellular protein is an RNA binding protein (RBP), a protein that binds single or double stranded RNA and contains one or more characteristic structural motifs. Examples of RBPs include stem-loop binding protein (SLBP), iron-responsive element binding protein-2 (IREB-2), and SECIS binding protein (SBP2).

In certain embodiments, the invention described herein relates to a post-transcriptional regulatory mechanism called “structurally interacting RNA” (or sxRNA). sxRNA is a trans-mRNA/miRNA interaction that forms a three way junction (3WJ). Certain features of this interaction can be used to design highly stable RNA constructs that inhibit non-coding RNAs (e.g., that act as anti-miRs), for example, by using a stem-loop instead of a bulge in the middle of an anti-miR RNA construct. This approach has three advantages for use as an anti-miR: (1) the presence of a stem-loop prevents cleavage of the anti-miR by the RISC complex, (2) many of the interactions we have informatically and experimentally observed have flanks of a stem-loop that are perfectly complementary to a miRNA without recruiting the RISC complex, and (3) importantly, three way junctions appear to be very stable structures.

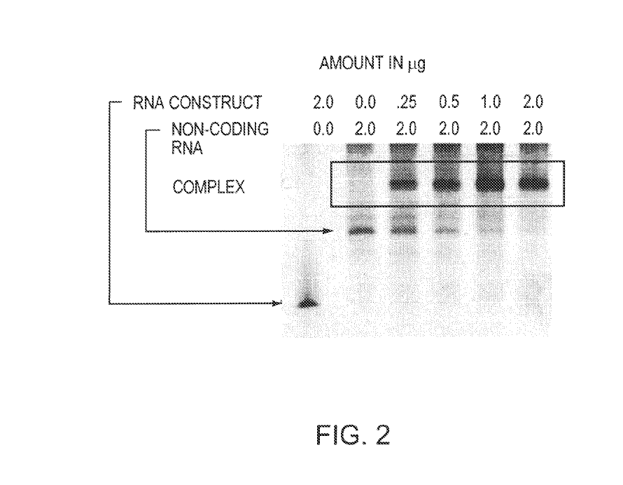

The stability of three way junctions might not be readily apparent. However, integral functional RNA based molecules such as ribosomal RNA would not be effective unless three way junctions were stable enough to hold them in a particular conformation the vast majority of the time. In studying three way junctions Zhang et al. ((2013) “Crystal structure of 3WJ core revealing divalent ion-promoted thermostability and assembly of the Phi29 hexameric motor pRNA,” RNA 19:1226-1237) found that DNA-DNA or DNA-RNA based three way junctions were moderately stable, but RNA-RNA based three way junctions were so strong that they remained bound, even in an 8 M urea denaturing gel. Similar results, shown in FIG. 2, indicate that, in certain embodiments, the interaction between RNA constructs of the present invention and non-coding RNA are so strong that they remain bound, even when heated to 95° C. in denaturing buffer and run on a TBE-Urea gel.

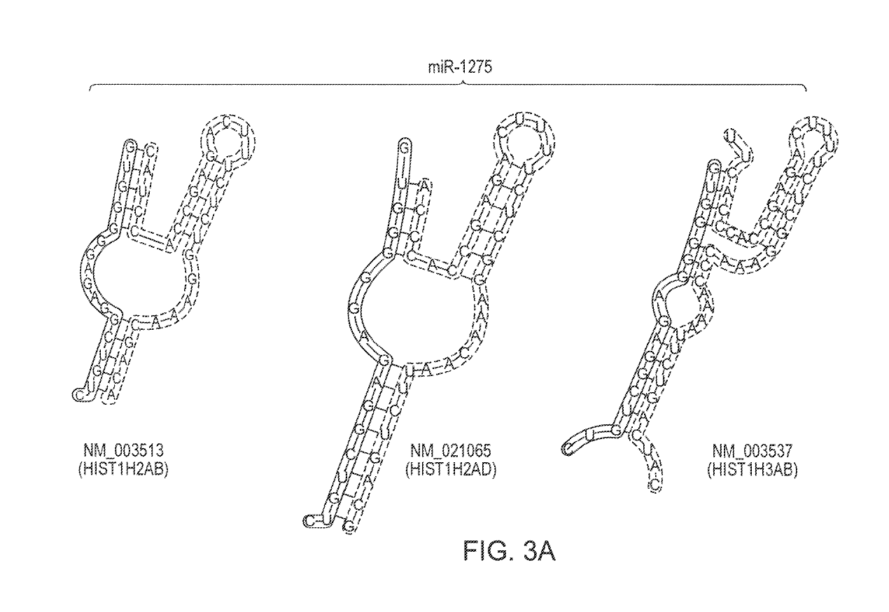

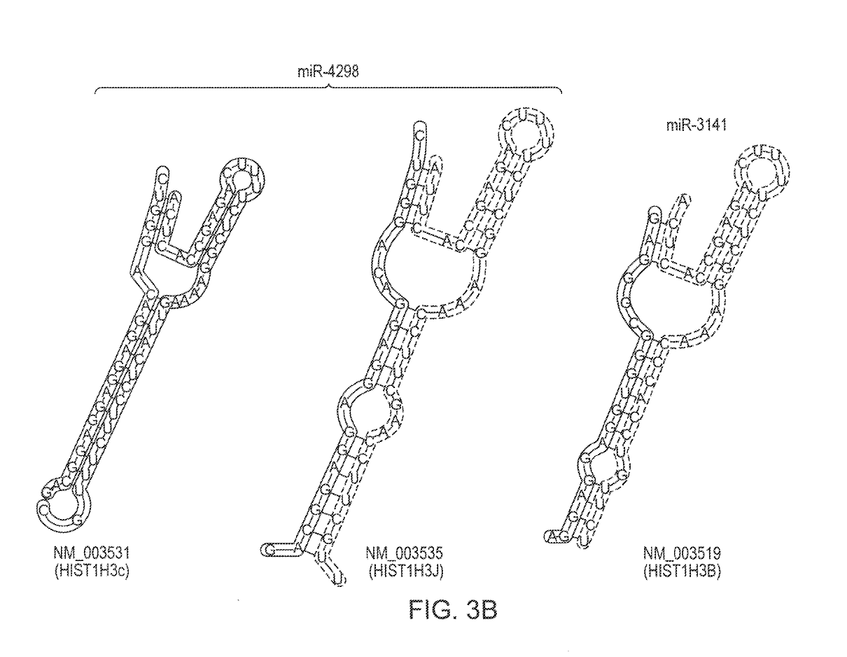

Making RNA constructs (e.g., anti-miRs) against specific non-coding RNAs (e.g., miRNAs) is described herein. RNA constructs comprise at least one stem-loop structure, but no specific stem-loop is preferred. Examples described herein predominantly use a histone stem-loop, but any stem-loop can be used to create an RNA construct. The stem can be long or short, and the sequence can be varied as long as the sequences will form a stem in the proper confirmation. The loop itself also can be varied, and can range from a hairpin structure to a complex loop comprising a variety of secondary structure including additional loops, cloverleafs, etc., comprising up to thousands of bases, as long as a non-coding RNA can preferentially bind the RNA construct as compared to a natural target. In certain embodiments, the stem-loop in its final conformation optionally can be a binding site for a cellular protein (e.g., an RBP), depending upon the desired effect. The stem-loop can also be incorporated anywhere between the start and end of the nucleotides complementary to the non-coding RNA with at least one complementary nucleotide on both sides of the stem-loop. The flanks of the stem-loop need to be generally complementary to the non-coding RNA (e.g., a miRNA target, however, as can be seen in FIG. 3, perfectly complementary sequences may not be required or desired. The flanks can each individually be the same length as the miRNA, longer than the miRNA extending past the complementary nucleotides or shorter than the miRNA and not complementing the miRNA to the end of the miRNA. There may be additional nucleotides on either side of the stem-loop between the stem-loop and the start of the complementary sequence.

Use of an RNA construct described herein can be used in combination with other strategies known in the art for inhibiting non-coding RNAs to increase efficacy. For example, use of modified nucleic acid bases can be used to achieve high affinity and/or stability by inhibiting catalytic cleavage of an RNA construct bound by the non-coding RNA (e.g., a miRNA) or by inhibiting other cellular nucleases. Examples of modified nucleic acids include phosphate backbone modification (e.g., phosphodiester or phosphothiorate internucleotide linkages), sugar modifications (e.g., locked nucleic acids (LNAs), 2′OMe, 2′-fluoro RNA (2′F) and 2′MOE), and non-ribose backbones (e.g., phosphoramidate morpholino oligo (PMO) and peptide nucleic acid (PNA)). See, Lennox et al. (2001) “Chemical modification and design of anti-miRNA oligonucleotides,” GENE THERAPY 18:1111-1120.

Other modifications or additions can be made to the non-coding RNA such as the addition of a biotin tag or fluorescent tag, or other changes generally used in the art.

### II. RNA Construct

A. General

The RNA construct comprises structural elements that aid in the binding of the non-coding RNA to the RNA construct such that the non-coding RNA binds preferentially to the RNA construct as compared to a natural target (e.g. an mRNA). Specifically, the RNA construct typically contains nucleotides that will lack binding partners at joining regions formed when bound to a non-coding RNA, providing flexibility which enhances the affinity with which the RNA construct binds to a non-coding RNA and/or the stability of the RNA construct-non-coding RNA complex. Modified nucleic acid bases, described above, can be used in the constructs described herein.

B. Uses

1. Inhibition of miRNA

An RNA construct can be designed to bind to a non-coding RNA, wherein the non-coding RNA binds to the RNA construct preferentially as compared to a natural target. Inhibition of the non-coding RNA typically results in the opposing effect of the function of the non-coding RNA. For example, inhibition of an miRNA can lead to increased production of the miRNA's natural target. In certain embodiments, an miRNA that normally promotes cellular functioning (e.g., by suppressing translation of an apoptotic protein) can be inhibited to kill the cell (e.g., an infected cell). This strategy can be used to target any pathogen, including viruses, bacteria, fungi or protozoa. Thus, the strategy can kill the cell harboring a pathogen and/or the pathogen itself. Beyond pathogens, this strategy can be used to eliminate diseased cells, such as neoplastic cells (e.g., cancer cells). Alternatively, an miRNA that normally inhibits cellular functioning (e.g., by suppressing translation of an inhibitor of apoptosis) can be inhibited to promote survival of the cell (e.g., a neurodegenerating cell).

2. Inhibition of miRNA and a Cellular Protein

In certain embodiments, RNA constructs comprise a binding site for a cellular protein (e.g., an RBP). The binding site may be present in the presence or absence of a non-coding RNA, or may form only when a non-coding RNA binds the RNA construct and stabilizes the binding site for the cellular protein. Binding of the cellular protein can inhibit the function of the cellular protein. In certain embodiments, inhibition of the cellular protein occurs because the cellular protein is sequestered by the RNA construct and therefore unable to function because it cannot make the necessary binding contacts or because it is mis-localized.

In certain embodiments, an RNA construct inhibits both a non-coding RNA and a cellular binding protein, leading to a certain functional output Inhibition of a non-coding RNA and a cellular protein can, for example, lead to the death of the cell. In certain embodiments, an RNA construct binds to and inhibits a non-coding RNA in an infected cell, and binds to and inhibits a cellular protein necessary for any essential cellular function (e.g., transcription and/or translation) Inhibition of both the non-coding RNA and the cellular protein leads to the death of the infected cell. In certain embodiments, inhibition of both the non-coding RNA and the cellular protein leads to the synergistic inhibition of a cellular function than inhibition of either the non-coding RNA or the cellular protein alone.

C. Construct Design

The following section describes how to design an RNA construct of the invention. RNA constructs comprise at least one stem-loop structure, but no specific stem-loop is preferred. Examples described herein predominantly use a histone stem-loop, but any stem-loop can be used to create an RNA construct. The stem can be long or short, and sequence can be varied as long as the sequences will form a stem in the proper confirmation. The loop itself also can be varied, and can range from a hairpin structure to a complex loop comprising a variety of secondary structure including additional loops, cloverleafs, etc., comprising up to thousands of bases, as long as a non-coding RNA can preferentially bind the RNA construct as compared to a natural target. In certain embodiments, the stem-loop in its final conformation optionally can be a binding site for a cellular protein (e.g., an RBP), depending upon the desired effect. The stem-loop can also be incorporated anywhere between the start and end of the nucleotides complementary to the non-coding RNA with at least one complementary nucleotide on both sides of the stem-loop.

The flanks of the stem-loop need to be generally complementary to the non-coding RNA (e.g., a miRNA target), however, as can be seen in FIG. 3, perfectly complementary sequences may not be required or desired. The flanks can each individually be the same length as the miRNA, longer than the miRNA extending past the complementary nucleotides or shorter than the miRNA and not complementing the miRNA to the end of the miRNA. There may be additional nucleotides on either side of the stem-loop between the stem-loop and the start of the complementary sequence.

While some details of design will depend upon the stem-loop structure, and its corresponding mode of action, typically design of RNA constructs will involve the following steps (which need not be performed in the order listed).

### 1. Design of Three-Way Junctions

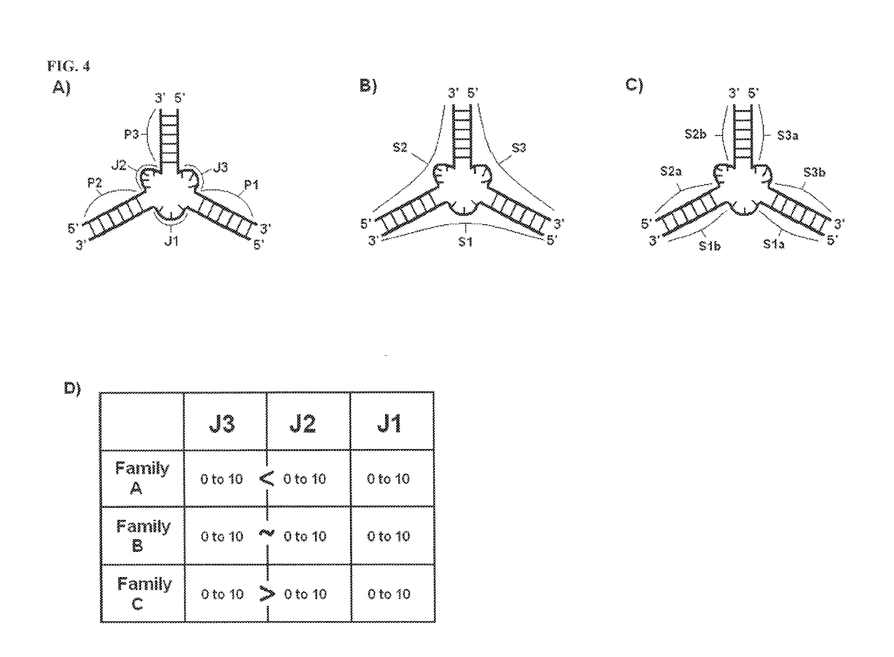

Three-way junctions are found in nature. (See, e.g., Lescoute et al. (2006) RNA 12:83-93.) The present invention simulates these natural structures and utilizes these simulated three-way junctions as a pattern upon which to build an RNA construct. FIGS. 4A-C provide the nomenclature of the RNA constructs patterned on three-way junctions. This nomenclature will be used throughout the present patent application. FIG. 4A shows a three-way junction having three base-paired stems (P1, P2, and P3) of variable length and three corresponding joining regions (J1, J2, and J3). The joining regions each independently can be from 0 to about 10 unpaired nucleotides, for example from about 2 to about 6 unpaired nucleotides. FIG. 4B shows the three-way junction of FIG. 4A, indicating the positions of the three RNA molecules of the junction, S1 through S3. S1 is the strand that connects P1 to P2 through J1, S2 is the strand that connects P2 to P3 through J2, and S3 is the strand that connects P3 to P1 through J3. FIG. 4C further divides the strands S1-S3 into 5′ to 3′ segments labeled “a” and “b.” For example, S1 is divided into two segments, S1a being the segment of P1 leading into the J1 joining region and S1b being the segment of P2 leading out of the J1 joining region. S2 and S3 are each similarly divided into “a” and “b” segments.

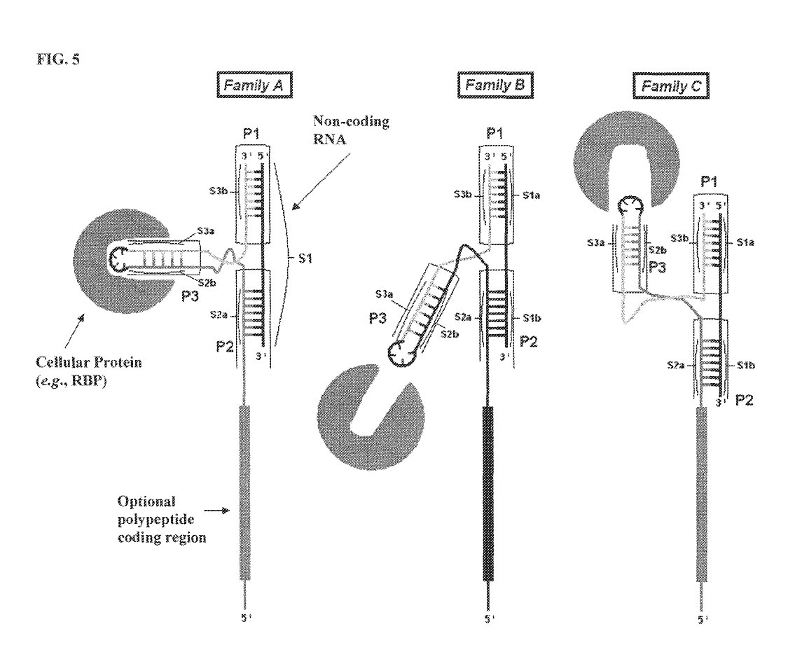

When developing an RNA construct, the joining regions of the construct as well as the non-coding RNA are chosen according to FIG. 4D. Depending upon how the joining regions are chosen, one can design a construct from one of three families, Families A, B, or C (FIG. 4D). In embodiments in which a cellular protein binds the stem-loop of the RNA construct, the choice of three-way junction family will be governed by which of these conformations will not obstruct the activity (e.g., protein binding) of the stem-loop. FIG. 5 depicts a generalized example of the family selection process, using a construct in which stem P3 forms the stem-loop and strand S1 is the non-coding RNA. In this example, the Family A structure was chosen because it allows a cellular protein of interest to associate with the stem-loop. Families B and C are not used in this example because P2 and P1, respectively, obstruct the cellular protein from binding to the stem-loop. In other examples, Family B or Family C might provide a better platform, depending upon the steric interactions of the cellular protein with the stem-loop.

If an RNA construct patterned on Family A is desired, J3 is selected to include any number of unpaired nucleotides from 0 to about 10 and J2 is selected to include any number of unpaired nucleotides, from 0 to about 10, such that the number of unpaired nucleotides selected for J3 is less that the number selected for J2. J1 includes any number of unpaired nucleotides from 0 to about 10. In certain embodiments, the number of unpaired nucleotides selected for J3 is between 1 and 3, the number of unpaired nucleotides in region J2 is between 3 and 9, and the number of unpaired nucleotides in region J1 is between 0 and 4. If an RNA construct patterned on the Family B conformation is desired, J3 is selected to include any number of unpaired nucleotides from 0 to about 10 and should be approximately equal (e.g., equal to 0 to about 10) to the number selected for J2. J1 includes any number of unpaired nucleotides from 0 to about 10. In certain embodiments, the number of unpaired nucleotides in each region (J1, J2 and J3) is between 2 and 6. If an RNA construct patterned on Family C is desired, J3 is selected to include any number of unpaired nucleotides from 0 to about 10 and J2 is selected to include any number of unpaired nucleotides, from 0 to about 10, such that the number of unpaired nucleotides in J3 is greater than the number selected for J2. J1 includes any number of unpaired nucleotides from 0 to about 10. In certain embodiments, the number of unpaired nucleotides selected for J3 is between 3 and 9, the number of unpaired nucleotides in region J2 is between 1 and 5, and the number of unpaired nucleotides in region J1 is between 0 and 5.

Tertiary interactions of bases in the joining regions with bases in the stem regions, as identified by crystallography in natural forms of three way junctions, may be engineered. On this basis, one can change the conformation to improve a given activity or increase affinity and/or stability.

### 2. Design of Optionally Weakened Stem-Loop

To design a weakened stem-loop, point mutations are engineered in the wild-type (including a consensus motif sequence) by introducing non-canonical base pairs (i.e., mismatches) so that structure formation, without a trans-acting support, is no longer energetically favorable. The resulting weakened structure typically should not form bonds strong enough to spontaneously establish an active form of the structure, and when structure is re-formed via trans-acting stabilization, the bond distances typically should not interfere with function. The positions and identity of nucleotides important for activity typically are identified so that these bases are not changed. While vital nucleotides typically are not altered, the nucleotide with which a given vital nucleotide pairs may be able to be altered to weaken the stem without sacrificing binding to the reconstituted structure. Stem nucleotides that only provide structural integrity can be altered to reduce thermodynamic favorability of stem formation. In this case, both bases of a pair can be altered if desired. Canonical base pairs are defined as A-U, U-A, G-C, C-G. There are a number of alternative pairings such as G-U and U-U with varying energy levels and bond distance. The choice as to which bases should be substituted is made with the awareness that if relative bond distance and/or conformation of certain bases in the stem is important to activity, appropriate non-canonical pairings should be chosen (See, for example, Leontis, et al. (2002) NUCLEIC ACIDS RESEARCH 30(16):3497-3531.) For example, to weaken a canonical cis Watson-Crick/Watson-Crick G-C base pair while preserving C1′-C1′ bond length distance (10.3 angstroms), a G-U base pair (10.2 angstroms) should be substituted rather than, for example, a U-U base pair (8.1 angstroms). Table 2 provides bond lengths of canonical and non-canonical cis Watson-Crick/Watson-Crick base pairings and allows for selection of appropriate pairings.

TABLE 2

| | C1′-C1′ |
|---|---|
| Pairing | distance |
| U-U | 8.1 Å |
| C-C | 8.5 Å |
| U-G | 10.2 Å |
| G-U | 10.2 Å |
| C-G | 10.3 Å |
| A-U | 10.3 Å |
| U-A | 10.3 Å |
| G-C | 10.3 Å |
| C-A | 10.4 Å |
| A-C | 10.4 Å |
| U-C | 11.8 Å |
| C-U | 11.8 Å |
| A-A | 12.3 Å |
| G-A | 12.5 Å |
| A-G | 12.5 Å |

In addition to bond distance, bond angles and their effects on helix twist and stem “kink” may be considered.

In the case of a cellular protein binding site, a consensus structure, which can be generated by binding assays, typically is the starting point, but any wild-type sequence (including any consensus sequence) can be used. Although the discussion of RNA construct design principles herein typically refers to an RNA sequence as the starting point, it should be understood that not only RNA sequences but also equivalent DNA sequences can be used. As the skilled artisan understands, an RNA sequence is equivalent to the sequence of a coding strand of genomic DNA or cDNA, because RNA that is transcribed from genomic DNA or cDNA has an identical sequence to the coding strand sequence of the genomic DNA or cDNA (with “T” nucleotides in the DNA sequence and “U” nucleotides in the RNA sequence). Thus, the design principles described herein are applicable when using RNA as a starting point or DNA as a starting point.

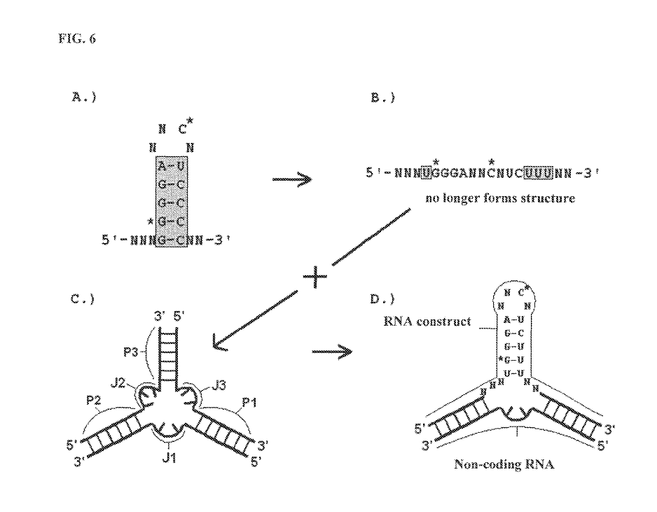

In order to demonstrate the concepts of the present invention, a generalized, mock stem-loop consensus sequence is depicted in FIG. 6A, with “N” denoting any nucleotide. Nucleotides in the paired stem portion of the structure, indicated by a shaded box, are mutated so that the structure can no longer spontaneously form. Nucleotides indicated by an asterisk are vital to cellular protein binding and therefore are not altered. FIG. 6B shows that a G to U mutation was made at the fourth nucleotide from the 5′ end, and C to U mutations were made for the three nucleotides at positions 3, 4 and 5, counting from the 3′ end. Each mutation depicted in FIG. 6B is indicated by a shaded box. Point mutations were made at spots calculated to increase minimum free energy (MFE) and reduce spontaneous formation of required secondary structure for cellular protein binding. FIG. 6D shows the structure formed by the binding of the weakened stem-loop structure to its non-coding RNA which follows the pattern of the three-way junction structure depicted in FIG. 6C. In this instance, J3 is 2, J2 is 3, and J1 is 2. Once the weakened stem-loop has been reinforced by the binding of the non-coding RNA, the relevant cellular protein can associate with the RNA construct.

### 3. Design of Flank Regions

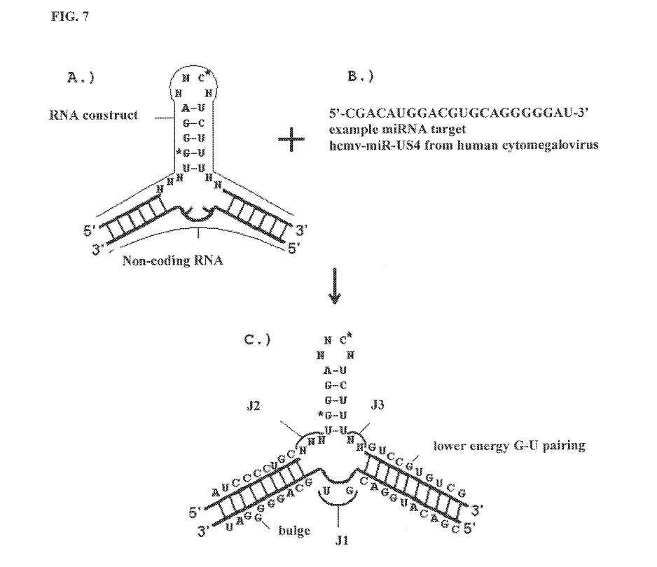

Flank regions are the regions of nucleotides adjacent to the stem on the 5′ and 3′ sides of the stem-loop structure that form two of the three joining regions and that bind to the non-coding RNA. FIGS. 7A-C illustrate the design of the 3′ and 5′ regions that flank the stem-loop of the mock RNA construct of FIGS. 6A-D. FIG. 7A depicts the hypothetical stem-loop previously shown in FIG. 6D. FIG. 7B shows an example non-coding RNA, a naturally-occurring miRNA from human cytomegalovirus, hcmv-miR-US4. In this example, the “N” nucleotides directly adjacent to the stem portion of the RNA construct form the unpaired nucleotides of the joining region. The three unpaired Ns correspond to the J2 region and the two unpaired Ns correspond to the J3 region of the three-way junction. Because the J2 region contains more unpaired nucleotides than does the J3 region, a Family A structure will form. These unpaired nucleotides can be any nucleotide, provided that they do not hybridize to the unpaired U and G nucleotides that make up the J1 joining region formed by the non-coding RNA or otherwise disrupt the structure of the junction. The nucleotides that make up the flank regions of the RNA construct distal to the joining regions are selected to hybridize to the chosen non-coding RNA, as shown in FIG. 7C. Flank sequences should either have no specific nucleotide requirements, so that perfect pairing can be achieved, or those bases that must retain specific identity should fall in an simulated joining region, be a canonical match for the appropriate bases in the non-coding RNA or provide a beneficial mismatch to avoid a non-reversible hybridization with the non-coding RNA. Regardless of whether specific requirements for flank nucleotides exist, the strength of binding of the RNA construct to the non-coding RNA can be affected by introducing non-canonical base pairings (i.e., mismatches) (e.g., “lower energy G-U pairing,” FIG. 7C) or by leaving out a base in the flank such that a nucleotide in the non-coding RNA is left unpaired, forming a bulge (“bulge,” FIG. 7C).

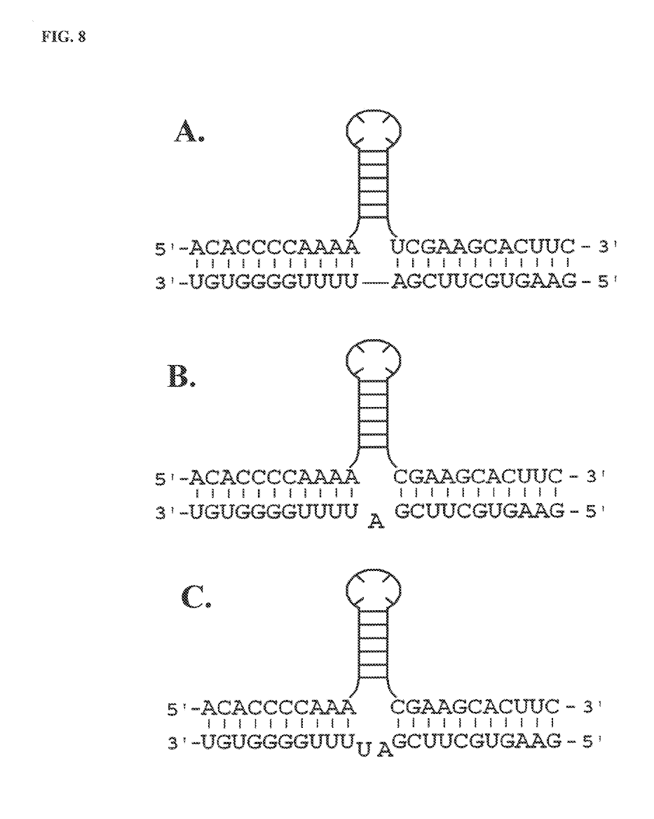

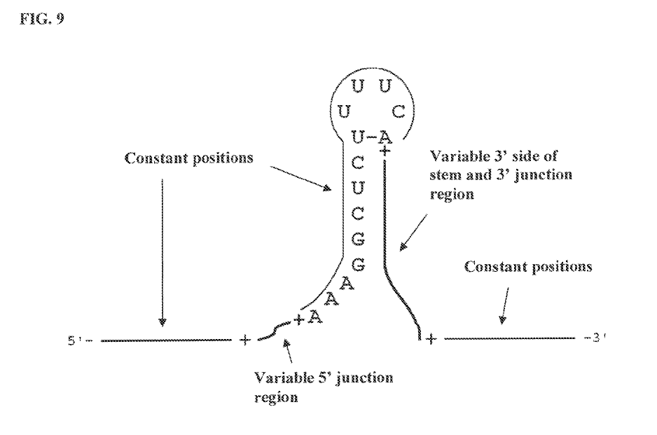

As shown in FIGS. 8A-C, the nucleotides chosen for the flank regions will dictate the size of the joining region defined by the non-coding RNA sequence. Again, these figures are intended to illustrate the principles of the invention. In FIG. 8A, the flank sequences are designed to pair with each nucleotide of the non-coding RNA sequence, forming a junction size of zero. In FIG. 8B, the flank sequences are designed such that one nucleotide of the non-coding RNA sequence is left without a binding partner, forming a junction size of one. FIG. 8C shows an example of flank sequences designed to leave two nucleotides of the non-coding RNA sequence without binding partners, forming a junction size of two.

Design of an RNA construct can be aided by use of any number of existing informatic RNA folding programs such as mfold, Sfold and the Vienna software. In addition, RNAFold can be used to examine optimal folds of a single RNA molecule; RNACofold can be used to examine optimal folds of two interacting RNA molecules; MiRanda or RNAHybrid can be used as part of high throughput screens to find naturally occurring instances of RNA construct-non-coding RNA interactions; and JMol can be used for visualizing existing crystal structures. These tools predict the probable structure of RNA and can be used in the design of complementary RNA constructs with predictable structural influences upon contact with the non-coding RNA. Depending on the non-coding RNA sequence, structural aspects may be exploited in the design of the RNA construct, especially with respect to areas of uniqueness in the non-coding RNA. Examples of this strategy include hairpin, stem-loops, cloverleafs, kissing complexes and other conformational structures known to result from traditional RNA-RNA interactions.

### 4. SELEX Approach

Generally, SELEX (Systematic Evolution of Ligands by Exponential Enrichment) is a method for generating a ligand of interest by taking a population of randomly-generated ligands (e.g., small molecules, polynucleotides or polypeptides) and systematically selecting and amplifying ligands that meet chosen criteria. To select for a ligand of interest, a very large polynucleotide library of random sequences is synthesized. Next, the sequences are exposed to a target binding partner (e.g., a protein or small molecule). Unbound sequences are removed, and the bound sequences are eluted and amplified. The amplified population is again exposed to the target binding partner, and the process is repeated. The stringency of binding and elution conditions can be increased to preferentially enrich only the tightest binding sequences. After a number of cycles, the remaining sequences are identified.

An RNA construct can be produced using a combination of the design approach outlined above and a SELEX-like approach. Use of a SELEX-like approach allows efficient screening of a large range of all possible RNA construct sequences for the best functional candidates. Designing an RNA construct with a SELEX-like approach has several steps in common with the above-described design approach, including choosing a functional motif for the RNA construct (e.g., histone stem-loop); identifying nucleotides important for desired activity (e.g., binding to a cellular protein); choosing a non-coding RNA sequence, such as an miRNA (e.g., HCMV-miR-US4 or miR-373), which is specific to a desired environment (e.g., HCMV-infected cells or breast cancer cells); and designing the flank sequences of the RNA construct by choosing the region of the non-coding RNA sequence to which the RNA construct will bind.

A SELEX-like approach may be taken by keeping the flank sequences and nucleotides critical to the functioning of the motif constant and randomly generating sequences for the remaining positions. Specifically, a library of nucleic acids is generated having sequences in which the constant positions are the same in each sequence, and random nucleotides are placed into the variable positions. FIG. 9 depicts an example of this approach using the histone stem-loop motif. In FIG. 9, three regions of nucleotides are kept constant during the SELEX procedure: a region that includes nucleotides required for the stem-loop binding protein (SLBP) to bind, as well as the two flank regions that correspond to the non-coding RNA sequence that has been chosen. FIG. 9 also depicts the two regions that are varied with randomly generated sequences: the 5′-most joining region (bold line flanked by “+” signs) and the right side of the stem to the 3′-most joining region (bold line flanked by “+” signs). The 5′ most joining region can be 0-7 nucleotides long, which will produce a joining region of 3-10 bases including the three adenines directly 3′ to the stem. The 3′ side of the stem and 3′-most joining region can be 5-15 nucleotides long. This region should include at least 5 nucleotides, so that each base of the 3′ side of the stem will be paired, and can include up to 10 nucleotides to make up the 3′ most joining region.

A starting library of potential RNA constructs having sequences in which constant positions are kept constant, and variable regions containing random sequences, is initially screened. In the initial screen, nucleic acids having sequences that bind to a protein of interest (e.g., a cellular binding protein) in the absence of the non-coding RNA are removed. Then, remaining nucleic acids are exposed to the protein of interest in the presence of the non-coding RNA, and unbound nucleic acids are removed. Bound nucleic acids are eluted, amplified, and the process is repeated under desired stringency conditions. Nucleic acids that remain after multiple rounds of selection are identified and further tested for desired activity. For example, identified nucleic acids may be attached to a polypeptide coding region that includes one or more reporter genes, placed in an expression environment in the presence or absence of the non-coding RNA and assayed to determine whether the reporter gene is expressed. Desired RNA constructs will be those that express the reporter gene only in the presence of the non-coding RNA.

An expression environment is any environment in which a polypeptide coding region can be expressed (e.g., a naturally-occurring cell, such as a neoplastic cell or infected cell; a non-naturally occurring cell; or a cell-free expression system (e.g., available from Life Technologies, Foster City, Calif.), such as rabbit reticulocyte lysate, wheat germ extract or E. coli cell-free system).

D. Histone Stem-Loop (HSL)

### 1. HSL Biology and Sequences

The principles of RNA construct design can be applied to a histone stem-loop starting point. For example, a wild-type histone stem-loop (HSL) motif (including a consensus sequence of a HSL motif) can be used as the starting point for further design of an RNA construct in accordance with the principles described above.

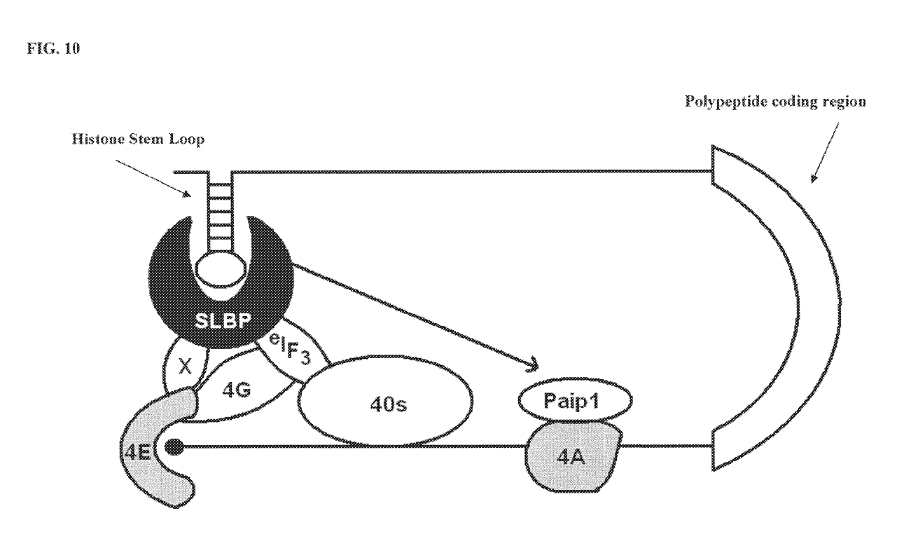

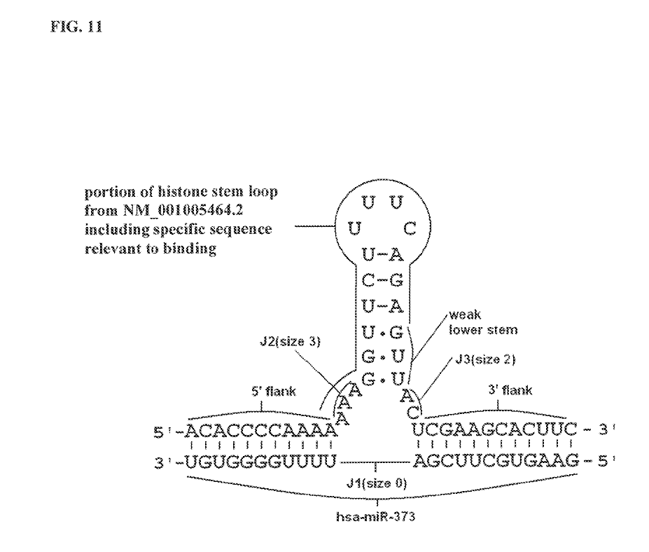

Metazoan cell cycle-regulated histone mRNAs are the only known cellular mRNAs that do not terminate in a poly(A) tail. Instead, these messages terminate in a conserved HSL motif, which functions to increase translation. The HSL motif is recognized and bound by “Stem-loop Binding Protein” (SLBP), an RBP, which is believed to upregulate the translation of one or more adjacent polypeptide coding regions through the action of a protein complex, shown in FIG. 10 (see, Gorgoni et al. (2005) RNA 11:1030-1042). As illustrated in FIG. 10, the SLBP binds the HSL in the 3′ UTR of the encoded polypeptide. Without wishing to be bound by the theory, the SLBP may interact with eukaryotic initiation factor 3 (eIF3) and other factors, represented by protein “X,” that interact with eIF4E (“4E” in FIG. 10) and eIF4G (“4G” in FIG. 10), which in turn binds to the 5′ cap (black circle). The formation of this complex may aid in the recruitment of the small ribosomal subunit (40s). In addition, the SLBP may directly bind Paip1, a protein thought to act in a manner similar to eIF4E, which binds eIF4A (“4A” in FIG. 10) to mediate translation. Alternatively, Paip1 simply may act to stabilize the complex.

The HSL motif has a conserved size and structure. The visual depiction of this is shown in the following HSL consensus sequence.

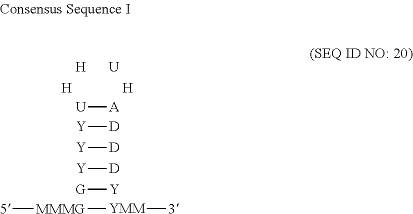

Table 3 shows the FASTA nucleic acid codes that are used throughout the description.

| FASTA code | Nucleotide |
|---|---|
| A | Adenosine |
| C | Cytidine |
| G | Guanine |
| T | Thymidine |
| U | Uridine |
| R | G A (purine) |
| Y | T U C (pyrimidine) |
| K | G T U (keto) |
| M | A C (amino) |
| S | G C (strong) |
| W | A T U (weak) |
| B | G T U C |
| D | G A T U |
| H | A C T U |
| V | G C A |
| N | A G C T U (any) |
| — | Gap of indeterminate length |

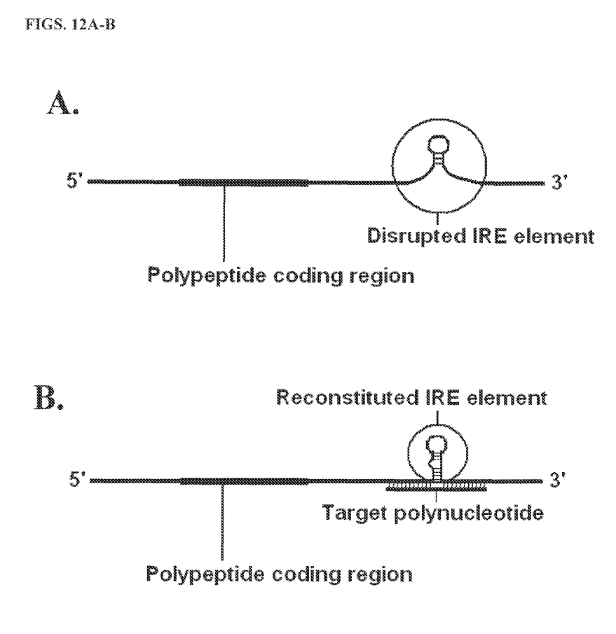

In the HSL, one or more of the guanines at the base of the 5′ side stem, the adenine on the 3′ side stem, and the first and third uridines in the loop are typically needed for binding to a SLBP. Accordingly, if SLBP binding is desired, care should be taken not to alter these bases when designing an RNA construct. However, in some instances the bases that are typically needed for binding to a SLBP may be altered even if some amount of activity is lost. The remaining nucleotides are more variable. Examples of naturally occurring HSL sequences are identified by GenBank number in FIG. 11 of U.S. Pat. No. 8,841,438, incorporated by reference herein, and can be accessed at www.ncbi.nlm.nih.gov/. “Start” and “end” indicate the location of the HSL sequence within the entire nucleotide sequence. Accordingly, any HSL consensus sequence or sequence from FIG. 11 of U.S. Pat. No. 8,841,438 can be used as the starting point to construct an RNA construct according to the invention.

While the HSL consensus sequence can be described visually, it can also be described using the logic search pattern (“PatSearch description”) described in Grillo, et al. (2003) NUCLEIC ACIDS RESEARCH 31(13)3608-3612.

The PatSearch description for the HSL described visually above is:
r1={AU,UA,GC,CG,GU,UG}
MMM p1=GGYYY U HHUH A r1˜p1 MM

“r1” indicates the rules for pairing to be applied to the consensus sequence. For example, the HSL stem can include “AU” base pairings, “UA” base pairings, “GC” base pairings, “CG” base pairings, “GU” base pairings, and “UG” base pairings. The next line shows the consensus sequence, beginning at the 5′ end with three “M” nucleotides. Next, “p1” indicates the first pattern of the consensus sequence, “GGYYY,” which represents the 5′ side of the stem. The next nucleotides in the sequence are UHHUHA, which represent the loop. Next, “r1˜p1” indicates that the pairing rules “r1” must be applied to the “p1” sequence to furnish the 3′ side of the stem. Finally, the 3′ end of the consensus sequence has two amino nucleotides, “MM.”

Other examples of HSL consensus sequences are shown below.

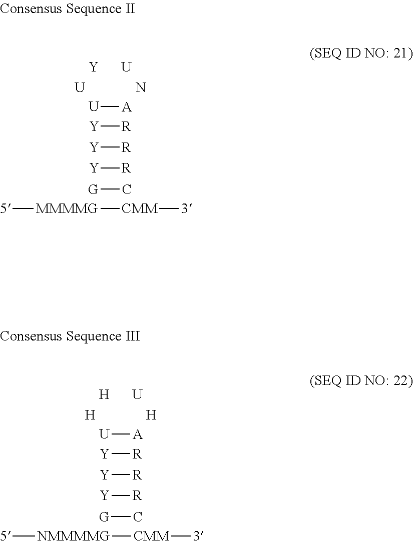

It should be understood that any wild-type HSL sequence (including a consensus sequence) can be used as the starting point to make any number of RNA constructs. From this wild-type HSL, at least one stem-weakening mutation is incorporated into the construct to decrease the formation of the stem-loop structure in the absence of a non-coding RNA. Consensus sequences that include non-canonical base pairings (i.e., mismatches) are considered to be weakened when such a pairing (e.g., a GU or a UG pairing) is chosen, even if the consensus sequence is broad enough to include non-canonical pairings at those same positions (e.g., because some wild-type sequences falling within the consensus sequence have canonical base pairing, rather than non-canonical base pairing, at the location). Alternatively, an RNA construct can contain a wild-type stem-loop if the stem-loop, in the context of the RNA construct, has an increased Minimum Free Energy (MFE) compared to the stem-loop in a wild-type context, such that the stem-loop of the RNA construct will be less stable than will the stem-loop in a wild-type context.

2. Design of an Example RNA Construct when Starting from an HSL Motif

This section illustrates the design of an RNA construct, shown in FIG. 11, starting from Consensus Sequence I.

a. Joining Regions

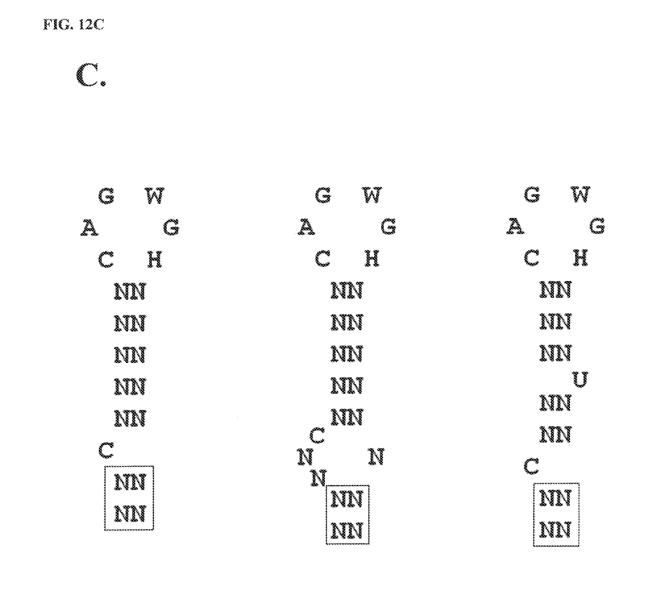

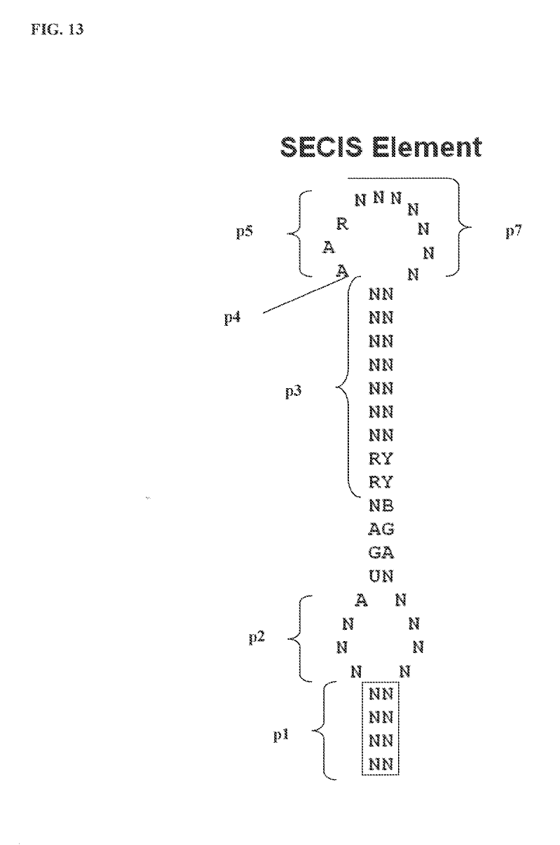

When designing an RNA construct based on an HSL motif, the size of the J2 joining region can be chosen so that the RNA construct-non-coding RNA structure forms a Family A, Family B or Family C structure. In the example depicted in FIG. 12, Family A is chosen. Family A likely allows the most favorable nucleic acid structure for binding to the SLBP. This is typically true of RNA constructs based upon the HSL structure.

HSL Consensus Sequence I begins with three “M” nucleotides. In FIG. 11, all three “M” nucleotides are chosen to be unpaired “A” nucleotides, thereby forming a J2 region of 3 nucleotides. However, the size of the joining region can be decreased by choosing all or some of the “M” nucleotides to be nucleotides that pair with the non-coding RNA. The size of the J2 region also can be increased by adding nucleotides 5′ to the “MMM” region that do not pair with the non-coding RNA.

The HSL consensus sequence ends with two “M” nucleotides. In FIG. 11, the two “M” nucleotides are chosen to be unpaired “AC,” thereby forming the J3 joining region. As with the J2 joining region, the size of the J3 joining region can be decreased by choosing all or some of the “M” nucleotides to be nucleotides that pair with the non-coding RNA, or it can be increased by adding nucleotides 5′ to the “MM” region that do not pair with the non-coding RNA. The J1 joining region in this example contains no nucleotides. Thus, this example has a Family A structure due to the choice of joining regions.

b. Optional Weakening of the Stem

The HSL consensus sequence has the sequence “GGYYY” on the 5′ side of the stem, corresponding to “GGUUC” in FIG. 12. Although the “r1” pairing rules are broad enough to include the non-canonical base pairings GU and UG in the stem, use of non-canonical base pairings (i.e., mismatches) at these positions, rather than canonical base pairs, can produce a weakened stem suitable for use with the present invention. Therefore, the choice of three GU base pairings in the stem, depicted in FIG. 11, weakens the stem to prevent formation of the stem-loop in the absence of the non-coding RNA.

c. Flank Regions

Flank regions can be designed according to the principles discussed above. The flank regions of the RNA construct include two of the three joining regions as well as sequence that is complementary to the non-coding RNA. In the example shown in FIG. 11, the flank regions include the three-way joining regions J2 (“AAA”) and J3 (“AC”), corresponding to “MMM and “MM,” respectively, in the consensus sequence. The nucleotides distal to the joining regions contain nucleotides that hybridize to the target sequence, which in this example is the human microRNA hsa-miR-373.

As described above, the selected HSL sequence is then further modified to attach the polypeptide coding region using standard molecular biological protocols.

E. Iron Response Element (IRE)

Alternatively, a wild-type Iron Response Element (IRE), including a consensus sequence of an IRE motif, can be used as the starting point for functional design of an RNA construct in accordance with the principles described above. The IRE is an stem-loop motif with a binding site for RBPs such as iron-responsive binding proteins, for example, aconitase 1 (ACO1, also known as iron-responsive element binding protein 1 or IRP1) and iron-responsive element binding protein 2 (IREB-2, also known as IRP2). (See Hentze et al. (1996) PROC. NATL. ACAD. SCI. USA, Vol. 93, pp. 8175-8182).

The structure of the IRE motif is conserved but the exact composition of nucleotides can be variable, providing a useful platform for design of an RNA construct. Three subtypes of IRE consensus sequences can be used as a starting point from which to design an RNA construct. FIG. 12C shows an example of each type of IRE consensus sequence. No specific requirements exist for the nucleotide composition of the paired bases of the stem (except that they must follow the “r1” pairing rules described below) or for the composition of the flank sequences. The lack of specific base requirements gives a large degree of flexibility for designing an RNA construct based on the consensus IRE sequences or specific naturally-occurring IRE sequences in accordance with the strategies outlined above. The boxed portions of the consensus sequences indicate the regions where the stem may be weakened by introducing non-canonical base pairings (i.e., mismatches). This lower stem region can be 2 to 6 nucleotides long in certain embodiments of the invention.

The PatSearch pattern for IRE subtype 1 is as follows:

1.) IRE1
r1={AU,UA,GC,CG,GU,UG}
p1=2 . . . 8 C p2=5 . . . 5 CAGWGH r1˜p2 r1˜p1

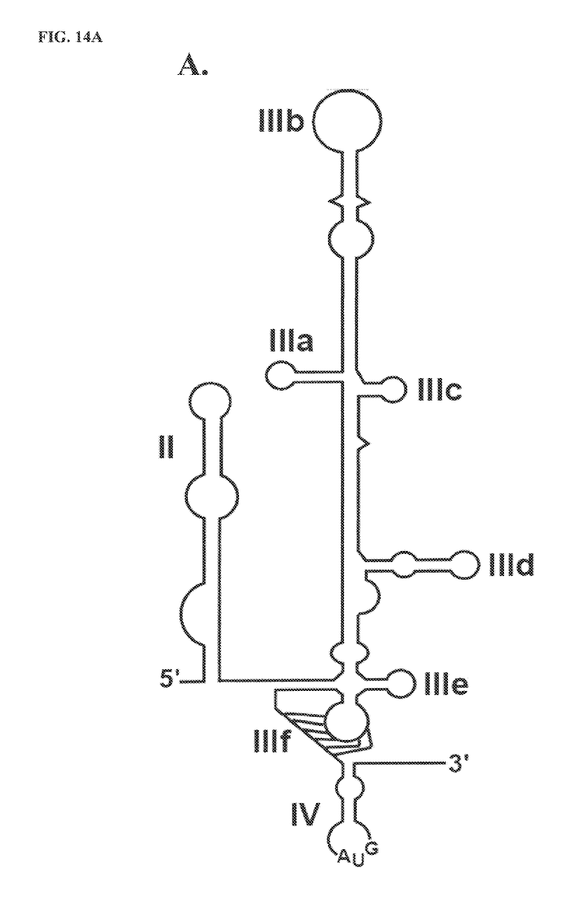

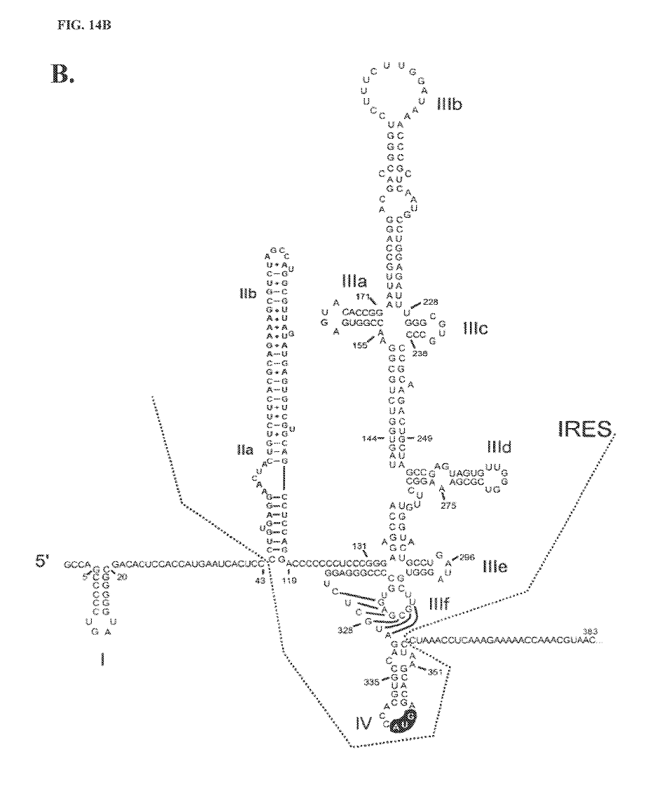

“r1” indicates the rules for pairing to be applied to the consensus sequence. For example, the IRE1 consensus sequence can include “AU” base pairings, “UA” base pairings, “GC” base pairings, “CG” base pairings, “GU” base pairings, and “UG” base pairings. The IRE1 consensus sequence begins at the 5′ end with the first pattern, “p1,” which forms the first part of the 5′ side of the stem. “p1” defines a string of 2 to 8 nucleotides of any composition (A, G, C, or U). The next part of the 5′ side of the stem is an unpaired “C” nucleotide, followed by the second pattern “p2,” the last part of the 5′ stem. “p2” defines a string of exactly 5 nucleotides of any composition (A, G, C, or U). Following “p2” is the loop, which consists of the sequence “CAGWGH.” Following the loop is the first part of the 3′ side of the stem, which can be any string of nucleotides that pairs to the “p2” pattern, provided that the nucleotides follow the “r1” rules for pairing (as indicated by the PatSearch description “r1˜p2”). The final part of the 3′ stem can be any string of nucleotides that pairs to the “p1” pattern, provided that the nucleotides follow the “r1” rules for pairing (as indicated by the PatSearch description “r1˜p1”). Examples of specific naturally-occurring IRE1 sequences are identified by GenBank number in FIG. 14 of U.S. Pat. No. 8,841,438, incorporated by reference herein, and can be accessed at www.ncbi.nlm.nih.gov/. “Start” and “end” indicate the location of the IRE1 sequence within the entire nucleotide sequence.

The PatSearch pattern for IRE subtype 2 is as follows:

2.) IRE2
r1={AU,UA,GC,CG,GU,UG}
p3=2 . . . 8 NNC p4=5 . . . 5 CAGWGH r1˜p4 N r1˜p3

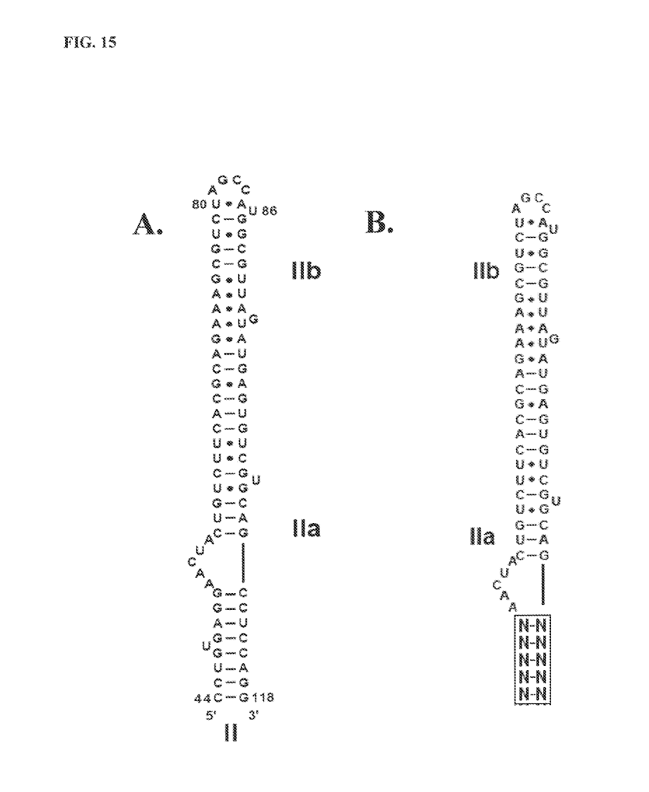

“r1” indicates the rules for pairing to be applied to the consensus sequence. For example, the IRE2 consensus sequence can include “AU” base pairings, “UA” base pairings, “GC” base pairings, “CG” base pairings, “GU” base pairings, and “UG” base pairings. The IRE2 consensus sequence begins at the 5′ end with the first pattern, “p3,” which forms the first part of the 5′ side of the stem. “p3” defines a string of 2 to 8 nucleotides of any composition (A, G, C, or U). The next part of the 5′ side of the stem is the sequence “NNC,” which will be unpaired. Next, the second pattern, “p4,” forms the last part of the 5′ stem. “p4” defines a string of exactly 5 nucleotides of any composition (A, G, C, or U). Following “p4” is the loop, which consists of the sequence “CAGWGH.” Following the loop is the first part of the 3′ side of the stem, which can be any string of nucleotides that pairs to the “p4” pattern, provided that the nucleotides follow the “r1” rules for pairing (as indicated by the PatSearch description “r1˜p4”). The second part of the 3′ stem can be any single unpaired nucleotide “N.” The final part of the 3′ stem can be any string of nucleotides that pairs to the “p3” pattern, provided that the nucleotides follow the “r1” rules for pairing (as indicated by the PatSearch description “r1˜p3”). Examples of specific naturally-occurring IRE2 sequences are identified in by GenBank number in FIG. 15 of U.S. Pat. No. 8,841,438, incorporated by reference herein, and can be accessed at www.ncbi.nlm.nih.gov/. “Start” and “end” indicate the location of the IRE2 sequence within the entire nucleotide sequence.

The PatSearch pattern for IRE subtype 3 is as follows:

3.) IRE3
r1={AU,UA,GC,CG,GU,UG}
p5=6 . . . 8 C p6=2 . . . 2 p7=3 . . . 3 CAGWGH r1˜p7 U r1˜p6 r1˜p5

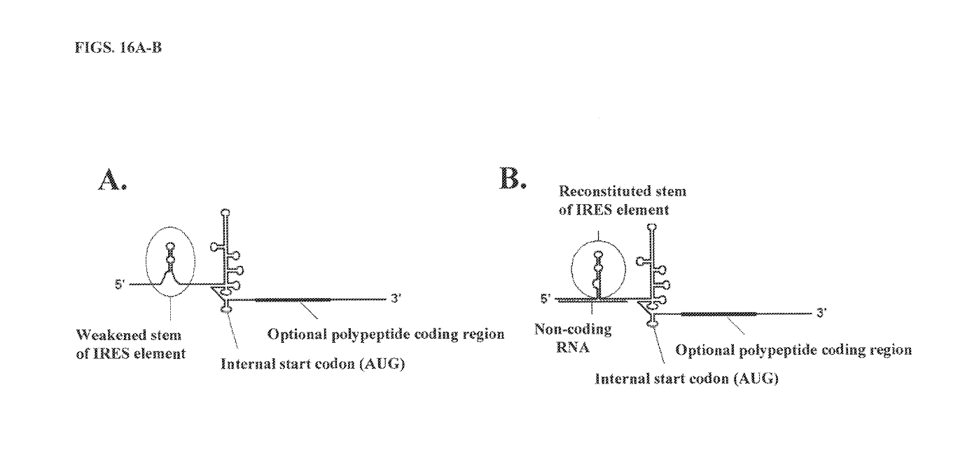

“r1” indicates the rules for pairing to be applied to the consensus sequence. For example, the IRE3 consensus sequence can include “AU” base pairings, “UA” base pairings, “GC” base pairings, “CG” base pairings, “GU” base pairings, and “UG” base pairings. The IRE3 consensus sequence begins at the 5′ end with the first pattern, “p5,” which forms the first part of the 5′ side of the stem. “p5” defines a string of 6 to 8 nucleotides of any composition (A, G, C, or U). The next part of the 5′ side of the stem is an unpaired “C” nucleotide, followed by the second pattern “p6,” which makes up the third part of the 5′ stem. “p6” defines a string of exactly 2 nucleotides of any composition (A, G, C, or U). The final part of the 5′ stem is the pattern “p7,” which consists of exactly 3 nucleotides. Following “p7” is the loop, which consists of the sequence “CAGWGH.” Following the loop is the first part of the 3′ side of the stem, which can be any string of nucleotides that pairs to the “p7” pattern, provided that the nucleotides follow the “r1” rules for pairing (as indicated by the PatSearch description “r1˜p7”). The second part of the 3′ stem is the unpaired nucleotide “U.” The next part of the 3′ stem can be any string of nucleotides that pairs to the “p6” pattern, provided that the nucleotides follow the “r1” rules for pairing (as indicated by the PatSearch description “r1˜p6”). The final part of the 3′ stem can be any string of nucleotides that pairs to the “p5” pattern, provided that the nucleotides follow the “r1” rules for pairing (as indicated by the PatSearch description “r1˜p5”). Examples of specific naturally-occurring IRE3 sequences are identified by GenBank number in FIG. 16 of U.S. Pat. No. 8,841,438, incorporated by reference herein, and can be accessed at www.ncbi.nlm.nih.gov/. “Start” and “end” indicate the location of the IRE3 sequence within the entire nucleotide sequence.

F. SECIS

Alternatively, a wild type Selenocysteine Insertion Element (SECIS), including a consensus sequence of a SECIS motif, can be used as the starting point for functional design of an RNA construct in accordance with the principles described above. Two varieties of SECIS elements exist, type 1 and type 2 (SECIS1, SECIS2). An active SECIS element present in the 3′UTR of an mRNA causes the translation machinery to reinterpret a UGA stop codon as a selenocysteine residue. Selenocysteine incorporation at UGA codons also uses specialized trans-acting factors. They include a selenocysteine-specific tRNA, an elongation factor specific for this tRNA and a SECIS-binding protein, (e.g., SBP2, which is an RBP), which recruits the elongation factor to the selenoprotein mRNA. Ribosomal Protein L30 also binds the SECIS element. It is believed that a complex of SELB (the specialized elongation factor), Sec-tRNA^{Sec} (selenocysteine tRNA), GTP (guanosine triphosphate), SBP2 and SECIS form and then associate with the ribosome via L30. At this point, a conformational change in the SECIS element triggers the release of Sec-tRNA^{Sec} and GTP hydrolysis, which allows incorporation of the selenocysteine residue into the polypeptide. (See, e.g., Chavatte et al. (2005) NATURE STRUCTURAL AND MOLECULAR BIOLOGY 12:408-418.)

The PatSearch description for the SECIS type 1 consensus sequence is as follows:

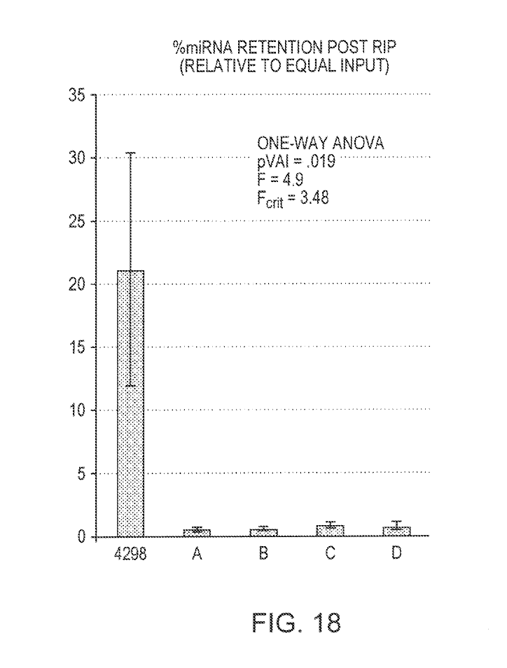

A.) SECIS1
r1={AU,UA,GC,CG,GU,UG}
p1=4 . . . 19
p2=2 . . . 9 R
UGAN
p3=8 . . . 12 p4=0 . . . 3 p5=aav p7=7 . . . 10 r1˜p3[1,0,0]
p3:(((̂RR|̂MC)|̂SU) 6 . . . 10)
NGAN
p8=2 . . . 9
r1˜p1
   “r1” indicates the rules for pairing to be applied to the consensus sequence. For example, the SECIS1 consensus sequence can include “AU” base pairings, “UA” base pairings, “GC” base pairings, “CG” base pairings, “GU” base pairings, and “UG” base pairings. The SECIS1 consensus sequence begins at the 5′ end with the first pattern, “p1,” which forms the first part of the 5′ side of the stem. “p1” defines a string of 4 to 19 nucleotides of any composition (A, G, C, or U). The next part of the 5′ side of the stem is the second pattern “p2,” which defines a string of 2 to 9 unpaired nucleotides of any composition (A, G, C, or U), followed by an “R.” The next part of the 5′ side of the stem is the string of nucleotides “UGAN.” The next part of the 5′ side of the stem is defined by the pattern “p3,” which consists of 8-12 nucleotides. “p3” is further defined by the PatSearch description, “p3:(((̂RR|̂MC)|̂SU) 6 . . . 10),” which indicates that “p3 must start with either “RR,” “MC,” or “SU” followed by 6-10 nucleotides of any composition (making up 8-12 nucleotides total). Pattern “p4” defines the 5′ end of the loop, which can be 0 to 3 nucleotides of any composition (A, G, C, or U). The loop continues with pattern “p5,” defined by the string of nucleotides “AAV.” The loop continues with pattern “p7,” which consists of 7 to 10 nucleotides of any composition (A, G, C, or U). Following the loop is the first part of the 3′ side of the stem, which can be any string of nucleotides that pairs to the “p3” pattern, provided that the nucleotides follow the “r1” rules for pairing. The PatSearch description, “r1˜p3[1,0,0]” indicates that the nucleotides in this section can, but are not required to, incorporate 1 mismatch, 0 insertions, and 0 deletions. The second part of the 3′ stem is the string of nucleotides “NGAN.” The next part of the 3′ stem consists of 2 to 9 unpaired nucleotides of any composition (A, G, C, or U). The final part of the 3′ stem can be any string of nucleotides that pairs to the “p1” pattern, provided that the nucleotides follow the “r1” rules for pairing. Examples of specific naturally-occurring SECIS type 1 sequences are identified by GenBank number in FIG. 18 of U.S. Pat. No. 8,841,438, incorporated by reference herein, and can be accessed at www.ncbi.nlm.nih.gov/. “Start” and “end” indicate the location of the SECIS1 sequence within the entire nucleotide sequence.

The PatSearch description for the SECIS type 2 consensus sequence is as follows:

B.) SECIS2
r1={AU,UA,GC,CG,GU,UG}
p1=4 . . . 19
p2=2 . . . 9 A
UGAN
p3=8 . . . 12 p3:(((̂RR|̂MC)|̂SU) 6 . . . 10)
p4=0 . . . 3 p5=AAV p6=11 . . . 14 p6:(0 . . . 1 p7=2 . . . 7 3 . . . 6 r1˜p7 0 . . . 3$)
(((r1˜p3[1,0,0]|r1˜p3[1,1,0])|r1˜p3[1,0,1])|r1˜p3[0,1,1])
NGAN
p10=2 . . . 9
r1˜p1

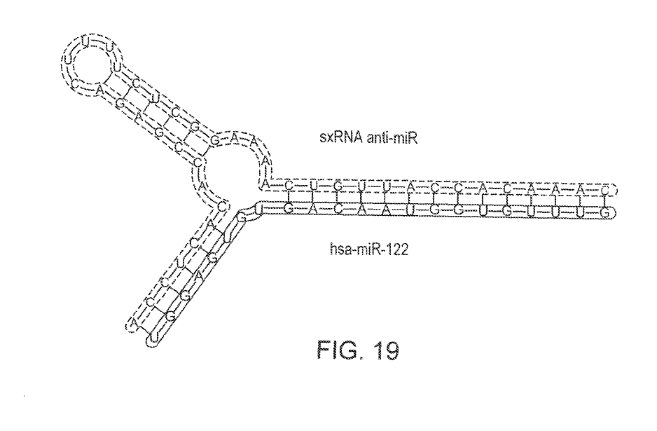

“r1” indicates the rules for pairing to be applied to the consensus sequence. For example, the SECIS2 consensus sequence can include “AU” base pairings, “UA” base pairings, “GC” base pairings, “CG” base pairings, “GU” base pairings, and “UG” base pairings. The SECIS2 consensus sequence begins at the 5′ end with the first pattern, “p1,” which forms the first part of the 5′ side of the stem. “p1” defines a string of 4 to 19 nucleotides of any composition (A, G, C, or U). The next part of the 5′ side of the stem is the second pattern “p2,” which defines a string of 2 to 9 unpaired nucleotides of any composition (A, G, C, or U), followed by an “A.” The next part of the 5′ side of the stem is the string of nucleotides “UGAN.” The next part of the 5′ side of the stem is defined by the pattern “p3,” which consists of 8-12 nucleotides. “p3” is further defined by the PatSearch description, “p3:(((̂rr|̂mc)|̂su) 6 . . . 10),” which indicates that “p3 must start with either “RR,” “MC,” or “SU” followed by 6-10 nucleotides of any composition (making up 8-12 nucleotides total). Pattern “p4” defines the 5′ end of the loop, and can be 0 to 3 nucleotides of any composition (A, G, C, or U). The loop continues with pattern “p5,” defined by the string of nucleotides “AAV.” The loop continues with pattern “p6,” which consists of 11 to 14 nucleotides which begins with 0 to 1 nucleotide of any compositions (A, G, C, or U), followed by the pattern p7, which can be 2 to 7 nucleotides of any composition (A, G, C, or U) followed by 3 to 6 nucleotides, provided that the last 3 nucleotides, which will form the first part of the 3′ side of the stem, follow the “r1” rules for pairing. The next part of the 3′ side of the stem can be any string of nucleotides that pairs to the “p3” pattern, provided that the nucleotides follow the “r1” rules for pairing. The PatSearch description, “(((r1˜p3[1,0,0]|r1˜p3[1,1,0])|r1˜p3[1,0,1])|r1˜p3[0,1,1])” indicates that the nucleotides in this section can (but are not required to) incorporate 1 mismatch, 0 insertions, and 0 deletions; 1 mismatch, 1 insertion, and 0 deletions; 1 mismatch, 0 insertions, and 1 deletion; or 0 mismatches, 1 insertion, and 1 deletion. The second part of the 3′ stem is the string of nucleotides “NGAN.” Pattern 10 defines the next part of the 3′ stem, which consists of 2 to 9 unpaired nucleotides of any composition (A, G, C, or U). The final part of the 3′ stem can be any string of nucleotides that pairs to the “p1” pattern, provided that the nucleotides follow the “r1” rules for pairing. Examples of specific naturally-occurring SECIS type 2 sequences are identified in by GenBank number in FIG. 19 of U.S. Pat. No. 8,841,438, incorporated by reference herein, and can be accessed at www.ncbi.nlm.nih.gov/. “Start” and “end” indicate the location of the SECIS2 sequence within the entire nucleotide sequence.

G. Internal Ribosome Entry Site (IRES)

Alternatively, a wild-type Internal Ribosomal Entry Site (IRES), including a consensus sequence of a IRES motif, can be used as the starting point for functional design of an RNA construct in accordance with the principles described above. The IRES is located in the 5′ UTR of mRNA and allows for 5′ cap-independent initiation of translation. Multiple forms have been identified and some interact with different factors to begin translation. For example, some IRESes bind eIF4G to initiate translation, while some act by directly binding to the 40S subunit. However, IRESes are not known to bind to an RBP. Instead, proper formation of the IRES facilitates binding of proteins that make up the translation machinery itself (e.g., eIF4G or the 40S subunit).

IRESes typically are large and more complex than a simple stem-loop. A few flaviviruses have an IRES with similar structure, shown generally in FIG. 14A. One example of an IRES of this type is the Hepatitis C virus IRES shown in FIG. 14B. One part of that structure, referred to as “domain II” in the Hepatitus C virus, can be used as a starting point for making an RNA construct (see FIG. 15A). FIG. 15A shows domain II (labeled IIa and IIb) from the Hepatitis C virus, and FIG. 15B depicts the region of nucleotides, labeled “N,” that can be modified to design an RNA construct. For example, nucleotides can be modified to weaken the stem, and flank nucleotides can be designed to match a non-coding RNA sequence. Examples of flavivirus IRESes with similar structure, including a domain II region in bold, are shown in FIG. 22 of U.S. Pat. No. 8,841,438, incorporated by reference herein. The sequences for other specific naturally-occurring IRES motifs from which an RNA construct can be designed are identified by GenBank number in FIG. 23 of U.S. Pat. No. 8,841,438, incorporated by reference herein, and can be accessed at www.ncbi.nlm.nih.gov/. “Start” and “end” indicate the location of the IRES sequence within the entire nucleotide sequence. Any of the sequences can by modified to produce an RNA construct.

### III. Non-Coding RNAs

Non-coding RNAs can be any endogenous or exogenously supplied RNA that does not normally lead to the expression of an encoded polypeptide and that binds to an RNA construct. Non-coding RNAs bind to the flank regions of an RNA construct, such that the non-coding RNA preferentially binds to the RNA construct as compared to a natural target (e.g., an mRNA). A non-coding RNA can be, but is not limited to, a viral polynucleotide, a bacterial polynucleotide, an polynucleotide expressed in a neoplastic cell, or a polynucleotide characteristic of a protein deficiency. Non-coding RNA can be found in, for example, but not limited to, a virally- or bacterially-infected cell, a neoplastic cell, a diseased cell, a tissue, a cell culture, or a sample containing polynucleotides (e.g., a blood sample).

The sequence of the non-coding RNA also should allow for the RNA construct to base pair with a favorable energy minimization to promote preferential binding, i.e., the non-coding RNA should bind preferentially to the RNA construct as compared to a natural target. For example, the calculated MFE (minimum free energy) structure should be such that the non-coding RNA is predicted to hybridize the RNA construct's flank regions as desired (rather than the non-coding RNA binding to a natural target, or the RNA construct folding with itself and the non-coding RNA folding with itself or the RNA construct and the non-coding RNA hybridizing in a manner other than what is desired).

### V. Production, Administration and Testing of an RNA Construct

### 1. Production of an RNA Construct

DNA encoding the RNA construct (and, optionally, a polypeptide coding region) of the present invention can be prepared in any number of ways known in the art. DNA fragments encoding portions of the RNA construct can be obtained from any source, for example, from a cDNA library or amplified from genomic DNA using polymerase chain reaction (PCR). Methods for isolating nucleic acids, synthesizing nucleic acids, hybridizing nucleic acids, making and screening cDNA libraries, and performing PCR are well known in the art (see, e.g., Gubler et al. (1983), GENE 25:263-269; Sambrook et al. (2nd ed. 1989) MOLECULAR CLONING, A LABORATORY MANUAL; Ausubel et al., eds. (1994) CURRENT PROTOCOLS IN MOLECULAR BIOLOGY), as are PCR methods (see U.S. Pat. Nos. 4,683,195 and 4,683,202; Innis et al., eds (1990) PCR PROTOCOLS: A GUIDE TO METHODS AND APPLICATIONS). Expression libraries are also well known to those of skill in the art. Additional basic texts disclosing the general methods of use in this invention include Kriegler (1990) GENE TRANSFER AND EXPRESSION: A LABORATORY MANUAL.

In an exemplary embodiment, at least a portion of a contemplated RNA construct is chemically synthesized. The single stranded molecules that comprise RNA constructs may be synthesized using any of a variety of techniques known in the art, such as those described in Usman et al. (1987) J. AM. CHEM. SOC. 109:7845; Scaringe et al. (1990) NUCLEIC ACIDS RES. 18:5433; Wincott et al. (1995) NUCLEIC ACIDS RES. 23:2677-2684; and Wincott et al. (1997) METHODS MOL. BIO. 74:59. The synthesis of oligonucleotides makes use of common nucleic acid protecting and coupling groups, such as dimethoxytrityl at the 5′-end and phosphoramidites at the 3′-end. As a non-limiting example, small scale syntheses may be conducted on an Applied Biosystems synthesizer using a 0.2 μmol scale protocol with a 2.5 min. coupling step for 2′-O-methylated nucleotides. Alternatively, syntheses at the 0.2 μmol scale may be performed on a 96-well plate synthesizer from Protogene (Palo Alto, Calif.). However, a larger or smaller scale of synthesis is also within the scope of the present invention. Suitable reagents for oligonucleotide synthesis, methods for RNA deprotection, and methods for RNA purification are known to those of skill in the art.

Alternative methods of chemical synthesis well known to the skilled artisan (see, for example, Engels et al. (1989) ANGEW. CHEM. INTL. ED. 28:716-734) include, for example, phosphotriester, phosphoramidite, and H-phosphonate methods for nucleic acid synthesis. Another method for such chemical synthesis is polymer-supported synthesis using standard phosphoramidite chemistry. Nucleic acids larger than about 100 nucleotides can be synthesized as several fragments using these methods. The fragments then can be ligated together to form the full length encoded RNA construct.

DNA encoding the RNA construct, or portions thereof, (and, optionally, a polypeptide coding region) can be inserted into an appropriate expression or amplification vector using standard ligation techniques. The vector is typically selected to be functional in the particular expression environment employed (i.e., the vector is compatible with the host cell machinery such that amplification of the RNA construct and/or expression of the RNA construct can occur). DNA encoding the RNA construct may be amplified/expressed in prokaryotic, yeast, insect (baculovirus systems) and/or eukaryotic host cells. These transgenes can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann et al. (1995) PROC. NATL. ACAD. SCI. USA 92:1292).

RNA constructs and, optionally, polypeptide coding regions can be expressed and purified using common molecular biology and biochemistry techniques. For example, recombinant expression vectors can be used which can be engineered to carry an encoded RNA construct and, optionally, polypeptide coding region into a host cell to provide for expression of the RNA construct and attached polypeptide coding region. Such vectors, for example, can be introduced into a host cell by transfection means including, but not limited to, heat shock, calcium phosphate, DEAE-dextran, electroporation or liposome-mediated transfer. Recombinant expression vectors include, but are not limited to, Escherichia coli based expression vectors such as BL21 (DE3) pLysS, COS cell-based expression vectors such as CDM8 or pDC201, or CHO cell-based expression vectors such as pED vectors. An RNA construct and, optionally, polypeptide coding region can be linked to one of any number of promoters in an expression vector that can be activated in the chosen cell line. In an embodiment, a cassette (RNA construct and promoter) is carried by a vector that contains a selectable marker such that cells receiving the vector can be identified.

For example, promoters to express the RNA construct within a cell line can be drawn from those that are functionally active within the host cell. Such promoters can include, but are not limited to, a T7 promoter, a CMV promoter, a SV40 early promoter, a herpes TK promoter, and others known in recombinant DNA technology. Inducible promoters can be used, and include promoters such as metallothionine promoter (MT), mouse mammary tumor virus promoter (MMTV), and others known to those skilled in the art. Exemplary selectable markers and their attendant selection agents can be drawn, for example, from the group including, but not limited to, ampicillin, kanamycin, aminoglycoside phosphotransferase/G418, hygromycin-B phosphotransferase/hygromycin-B, and amplifiable selection markers such as dihydrofolate reductase/methotrexate and others known to skilled practitioners.

Additional elements for directing the replication and transcription of an RNA construct (and, optionally, polypeptide coding region) can be included in a vector to express RNA constructs in a variety of cell types, including but not limited to, eukaryotic, prokaryotic, insect, plant and yeast. For example, microorganisms such as bacteria can be transformed with recombinant bacteriophage DNA, plasmid DNA or cosmid DNA expression vectors containing the RNA construct coding sequences; yeast can be transformed with recombinant yeast expression vectors containing the RNA construct coding sequences; insect cell systems can be infected with recombinant virus expression vectors (e.g., baculovirus) containing the RNA construct coding sequences; plant cell systems can be infected with recombinant virus expression vectors (e.g., cauliflower mosaic virus CaMV; tobacco mosaic virus, TMV) or transformed with recombinant plasmid expression vectors (e.g., Ti plasmid) containing the RNA construct coding sequences.

Typically, the vectors used in any of the host cells will contain at least a 5′ flanking sequence (also referred to as a promoter) and, optionally, other regulatory elements, such as an enhancer(s), an origin of replication element, a transcriptional termination element, a complete intron sequence containing a donor and acceptor splice site, a signal peptide sequence, a ribosome binding site element, a polyadenylation sequence, a polylinker region for inserting the DNA encoding the polypeptide coding region, and a selectable marker element. Suitable modifications may be made to the 5′ cap in order to direct translation of the encoded polypeptide using cap-independent mechanisms. Such modifications are well-known in the art (Kowalska, J. et al. (2008) NUCLEIC ACIDS SYMP. SER. 52:289-90).

The recombinant RNA construct expression vectors can be DNA plasmids or viral vectors. RNA construct-expressing viral vectors can be constructed based on, but not limited to, adeno-associated virus (for a review, see Muzyczka et al. (1992) CURR. TOPICS IN MICRO. AND IMMUNOL. 158:97-129), adenovirus (see, for example, Berkner et al. (1988) BIOTECHNIQUES 6:616; Rosenfeld et al. (1991) SCIENCE 252:431-434; and Rosenfeld et al. (1992) CELL 68:143-155), or alphavirus as well as others known in the art. Retroviruses have been used to introduce a variety of genes into many different cell types, including epithelial cells, in vitro and/or in vivo (see for example Eglitis, et al. (1985) SCIENCE 230:1395-1398; Danos and Mulligan (1988) PROC. NATL. ACAD. SCI. USA 85:6460-6464; Wilson et al. (1988) PROC. NATL. ACAD. SCI. USA 85:3014-3018; Armentano et al., (1990) PROC. NATL. ACAD. SCI. USA 87:61416145; Huber et al. (1991) PROC. NATL. ACAD. SCI. USA 88:8039-8043; Ferry et al. (1991), PROC. NATL. ACAD. SCI. USA 88:8377-8381; Chowdhury et al. (1991), SCIENCE 254:1802-1805; van Beusechem. et al. (1992) PROC. NATL. ACAD. SCI. USA 89:7640-19; Kay et al. (1992) Human Gene Therapy 3:641-647; Dai et al. (1992) PROC. NATL. ACAD. SCI. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Recombinant retroviral vectors capable of transcribing the RNA constructs of the present invention can be produced by transfecting the recombinant retroviral genome into suitable packaging cell lines such as PA317 and Psi-CRIP (Comette et al. (1991) HUMAN GENE THERAPY 2:5-10; Cone et al., 1984, PROC. NATL. ACAD. SCI. USA 81:6349). Recombinant adenoviral vectors can be used to infect a wide variety of cells and tissues in susceptible hosts (e.g., rat, hamster, dog, and chimpanzee) (Hsu et al. (1992) J. INFECTIOUS DISEASE, 166:769), and also have the advantage of not requiring mitotically active cells for infection.

### 2. Testing an RNA Construct

a. Assays to Test Preferential Binding and/or Binding Strength

A DNA vector encoding an RNA construct, and optionally a polypeptide coding region can be transfected into desired cell culture lines or cell-free expression systems with a non-coding RNA, and tested to determine whether the non-coding RNA binds preferentially to the RNA construct as compared to a natural target. Preferential binding of the non-coding RNA to the RNA construct can be measured using any assay known in the art.

In certain embodiments, binding is measured by determining the reduction in free energy (kCal) that occurs when an RNA construct binds a non-coding RNA and comparing that value to the reduction in free energy that occurs when the non-coding RNA binds a natural target (e.g., an mRNA or portion thereof). Reduction in free energy can range from about 1-10,000 kCal and, as is known in the art, can vary depending upon the overall length of the RNA construct and/or non-coding RNA. In certain embodiments, the reduction in free energy that occurs when an RNA construct binds a non-coding RNA is at least about 10 kCal, at least about 25 kCal, at least about 50 kCal, at least about 100 kCal, at least about 1,000 kCal. In certain embodiments, the reduction in free energy that occurs when a non-coding RNA binds an RNA construct is at least 1% more, at least 2% more, at least 5% more, at least 10% more, at least 25% more, at least 50% more at least 75% more, at least 95% more, at least 100% more, at least 200% more, at least 500% more than the reduction in free energy that occurs when a non-coding RNA binds its natural target.

In one example, a native binding assay can be used to determine whether a non-coding RNA preferentially binds an RNA construct. In a native binding assay, RNA is dissolved in a buffer containing 10 mM Tris-HCl (pH 8.0) and 100 mM NaCl. The RNA construct and non-coding RNA are mixed in a 1:2 ratio and annealed at 85° C. for 3 minutes and gradually cooled to room temperature for about an hour. The resulting mixture is run on a native PAGE (15% TBE-PAGE) gel with appropriate controls (RNA construct alone and non-coding RNA alone) and MW standards. Bands with a lower electrophoretic mobility (i.e., a higher molecular weight) than either the RNA construct or non-coding RNA alone are most likely the RNA construct complexed with the non-coding RNA, indicating that the RNA construct is capable of binding the non-coding RNA.

Denaturing gels also can be used to determine whether a non-coding RNA preferentially binds an RNA construct. For example, a 1 M, 3 M, 7 M or 8 M denaturing urea gel can be used to test the strength of binding of an RNA construct to a non-coding RNA. Strength of the binding interaction between RNA construct and non-coding RNA correlates to the ability to detect an interaction between an RNA construct and non-coding RNA at increasingly high urea concentrations. For example, very strong binding between an RNA construct and non-coding RNA will be detectable at higher urea molarity (e.g., 7 M, 8 M) while relatively weaker binding between an RNA construct and non-coding RNA will be detectable at higher urea concentrations (e.g., 1 M, 3 M). For non-coding RNA that binds preferentially to an RNA construct as compared to a natural target, binding between non-coding RNA and RNA construct may be detectible at higher urea concentrations (e.g., 7 M, 8 M) than binding between the non-coding RNA and its natural target, which may, for example, be detectible at lower urea concentrations (e.g., 1 M or 3 M) or on a native gel.

In certain embodiments, RNA immunoprecipitation (RIP) can be used to determine whether a non-coding RNA preferentially binds an RNA construct. RIP can be performed by tagging any component of the interaction (RNA construct, non-coding RNA, or cellular protein), for example, with a His tag, allowing the components to combine (e.g., in a tube or in a cell), isolating the tagged component (e.g., with an anti-His antibody), precipitating any bound RNA, and analyzing bound RNA on, for example, a TBE-gel. Various RIP protocols are known in the art. See, e.g., Jain et al. (2011) “RIP-Chip analysis: RNA-Binding Protein Immunoprecipitation-Microarray (Chip) Profiling,” METHODS MOL. BIOL. 703:247-263; Jayaseelan et al. (2011) “RIP: an mRNA localization technique,” METHODS MOL. BIOL. 714:407-422.)

In certain embodiments, when an RNA construct with a weakened stem is used, binding of the non-coding RNA can be tested by determining whether the RNA construct forms a stem-loop and/or binds a cellular protein (e.g., an RBP). The ability of an RNA construct to form a stem-loop and bind an RBP in the presence of a target sequence can be tested by incorporating a label, for example, a radiolabel, fluorescent label, or luminescent label, into the RNA construct. The RNA construct, non-coding RNA and any additional desired reagents, such as buffers, may be added to the appropriate RBP. If a non-coding RNA is present, the RNA construct assumes a structure to which the RBP can bind. The RBP is then isolated and the presence of label is assayed. Detection of the label indicates that the RNA construct has attained the structure required for binding to the RBP, which further indicates that the non-coding RNA is present. Alternatively, assays useful for detecting the binding of an RNA construct to an RBP, but that do not require labeled RNA construct, include, but are not limited to, absorbance assays, immunoassays, assays for enzymatic activity of the RBP and Western blots.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum Dye®, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH-CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin EBG, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Other useful fluorescent labels include fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichlo-ro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including GE Healthcare Bio-Sciences, Piscataway, N.J.; Life Technologies, Grand Island, N.Y.; and Sigma Aldrich, St. Louis, Mo.

It is further contemplated that labels that detect a structural change in a polynucleotide can be incorporated into an RNA construct or directly into a nucleotide of the RNA construct to detect the conformational change that results upon binding to a non-coding RNA. Many such labels are known to those of skill in the art. Examples of labels suitable for use in the disclosed method are radioactive isotopes, fluorescent molecules, phosphorescent molecules, enzymes, antibodies, and ligands. More specifically, molecular beacons, Amplifluors®, FRET probes, cleavable FRET probes, TagMan® probes, scorpion primers, fluorescent triplex oligonucleotides including but not limited to triplex molecular beacons or triplex FRET probes, fluorescent water-soluble conjugated polymers, PNA probes or QPNA probes, for example, can be used to activate or quench a given label based on stem formation.

Stem activated labels are labels or pairs of labels where fluorescence is increased or altered by formation of a stem structure. Stem activated labels can include an acceptor fluorescent label and a donor moiety such that, when the acceptor and donor are in proximity (when the nucleic acid strands containing the labels form a stem structure), fluorescence resonance energy transfer from the donor to the acceptor causes the acceptor to fluoresce. Stem activated labels are typically pairs of labels positioned on nucleic acid molecules, such as the sides of the stem-loop, such that the acceptor and donor are brought into proximity when a stem structure is formed in the nucleic acid molecule. If the donor moiety of a stem activated label is itself a fluorescent label, it can release energy as fluorescence (typically at a different wavelength than the fluorescence of the acceptor) when not in proximity to an acceptor (that is, when a stem structure is not formed). When the stem structure forms, the overall effect would then be a reduction of donor fluorescence and an increase in acceptor fluorescence. FRET probes are an example of the use of stem activated labels, the operational principles of which can be adapted for use with RNA constructs.

Stem-quenched labels are fluorescent labels positioned on a nucleic acid such that when a stem structure forms a quenching moiety is brought into proximity such that fluorescence from the label is quenched. When the stem is disrupted, the quenching moiety is no longer in proximity to the fluorescent label and fluorescence increases. Examples of this effect can be found in molecular beacons, fluorescent triplex oligonucleotides, triplex molecular beacons, triplex FRET probes, and QPNA probes, the operational principles of which can be adapted for use with RNA constructs.

Examples of labels that can be incorporated into nucleic acids include nucleotide analogs such as BrdUrd (5-bromodeoxyuridine, Hoy et al., (1993) MUTATION RESEARCH 290:217-230), aminoallyldeoxyuridine (Henegariu et al., (2000) NATURE BIOTECHNOLOGY 18:345-348), 5-methylcytosine (Sano et al., (1988) BIOCHIM. BIOPHYS. ACTA 951:157-165), bromouridine (Wansick et al., (1993) J. CELL BIOLOGY 122:283-293) and nucleotides modified with biotin (Langer et al., (1981) PROC. NATL. ACAD. SCI. USA 78:6633) or with suitable haptens such as digoxygenin (Kerkhof (1992) ANAL. BIOCHEM. 205:359-364). Suitable fluorescence-labeled nucleotides are Fluorescein-isothiocyanate-dUTP, Cyanine-3-dUTP and Cyanine-5-dUTP (Yu et al. (1994) NUCLEIC ACIDS RES. 22:3226-3232). An exemplary nucleotide analog label for DNA is BrdUrd (bromodeoxyuridine, BrdUrd, BrdU, BUdR, Sigma-Aldrich Co.). Other useful nucleotide analogs for incorporation of label into DNA are AA-dUTP (aminoallyl-deoxyuridine triphosphate, Sigma-Aldrich Co.), and 5-methyl-dCTP (Roche Molecular Biochemicals). A useful nucleotide analog for incorporation of label into RNA is biotin-16-UTP (biotin-16-uridine-5′-triphosphate, Roche Molecular Biochemicals). Fluorescein, Cy3, and Cy5 can be linked to dUTP for direct labelling. Cy3.5 and Cy7 are available as avidin or anti-digoxygenin conjugates for secondary detection of biotin- or digoxygenin-labelled probes.

Labels that are incorporated into nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1³,7]decane]-4-yl) phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Methods for detecting and measuring signals generated by labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary label coupled to the antibody.

b. Assays to Test Inhibition of Non-Coding RNA

In certain embodiments, an RNA construct can inhibit the function of a non-coding RNA to which it binds. Inhibition may result from sequestration (i.e., the non-coding RNA is bound to the RNA construct and therefore not available for binding to its natural target) or Tests of inhibition of function can vary based upon the known function of the non-coding RNA. Generally, however, inhibition of a non-coding RNA by an RNA construct can be determined by (a) measuring the quantity of a downstream target of the non-coding RNA in the presence of the RNA construct; (b) measuring the quantity of the downstream target of the non-coding RNA in the absence of the RNA construct; and (c) determining that the quantity of the downstream target in the presence of the RNA construct is greater that the quantity of the downstream target the absence of the RNA construct. The downstream target can be, for example, an mRNA or a protein.

In certain embodiments, the non-coding RNA is an miRNA and the RNA construct inhibits the function of the miRNA as determined by (a) measuring the quantity of a downstream target of the miRNA in the presence of the RNA construct; (b) measuring the quantity of the downstream target of the miRNA in the absence of the RNA construct; and (c) determining that the quantity of the downstream target in the presence of the RNA construct is greater that the quantity of the downstream target the absence of the RNA construct. The downstream target can be, for example, an mRNA or a protein. In certain embodiments, an RNA construct reduces the function of a non-coding RNA by 1%, 2%, 5%, 10%, 15%, 25%, 50%, 75% 95% or 100% (completely eliminates function). For example, an RNA construct may, in an expression environment, reduce the quantity (e.g., concentration) of a downstream target (e.g., an mRNA or protein) of a non-coding RNA (e.g., an miRNA) by 1%, 2%, 5%, 10%, 15%, 25%, 50%, 75% 95% or 100% (as compared to the quantity of the downstream target in the expression environment in the absence of the RNA construct).

### 3. Administration of an RNA Construct

Any of the delivery methods known in the art suitable for use with RNA-based drugs are contemplated to work for RNA constructs according to the invention. For example, recombinant vectors capable of expressing RNA constructs are delivered as described above, and persist in target cells. Alternatively, viral vectors can be used that provide for transient expression of RNA constructs. Such vectors can be repeatedly administered as necessary. Once expressed, the RNA constructs bind to non-coding RNAs and undergo a conformational change, for example, strengthening of a stem-loop structure. Delivery of RNA construct-expressing vectors can be systemic, such as by intravenous or intramuscular administration, by administration to target cells ex-planted from a multicellular organism followed by reintroduction into the multicellular organism, or by any other means that allows for introduction into a desired target cell.

DNA plasmids carrying RNA constructs can be transfected into target cells as a complex with cationic lipid carriers (e.g. Oligofectamine) or non-cationic lipid-based carriers (e.g. Transit-TKO™). Multiple lipid transfections for RNA constructs targeting different regions of a single non-coding RNA or multiple non-coding RNAs over a period of a week or more are also contemplated by the present invention. Successful introduction of the vectors of the invention into host cells can be monitored using various known methods. For example, transient transfection can be signaled with a reporter, such as a fluorescent marker, such as Green Fluorescent Protein (GFP). Stable transfection of ex vivo cells can be ensured using markers that provide the transfected cell with resistance to specific environmental factors (e.g., antibiotics and drugs), such as hygromycin B resistance.

Alternatively, RNA constructs can be produced in cell culture and isolated, or can be chemically synthesized, and administered. Absorption or uptake of an RNA construct can occur through unaided diffusive or active cellular processes, or by auxiliary agents or devices. An RNA construct may be introduced into a cell, either in vitro or where the cell is part of a living organism. If the cell is part of an organism, introduction into the cell will include delivery to the organism. For example, for in vivo delivery, an RNA construct can be injected into a tissue site or administered systemically. In vitro introduction into a cell includes methods known in the art such as electroporation and lipofection.

An RNA construct can be chemically modified to enhance stability. The nucleic acids of the invention may be synthesized and/or modified by methods well established in the art, such as those described in CURRENT PROTOCOLS IN NUCLEIC ACID CHEMISTRY, Beaucage, S. L. et al. (Edrs.), John Wiley & Sons, Inc., New York, N.Y., USA, which is hereby incorporated herein by reference. Chemical modifications may include, but are not limited to 2′ modifications, introduction of non-natural bases, covalent attachment to a ligand, and replacement of phosphate linkages with thiophosphate linkages.

In yet another embodiment, nucleotides may be modified to prevent or inhibit the activation of cellular enzymes, such as, for example, without limitation, certain nucleases. Techniques for inhibiting the activation of cellular enzymes are known in the art including, but not limited to, 2′-amino modifications, 2′-amino sugar modifications, 2′-F sugar modifications, 2′-F modifications, 2′-alkyl sugar modifications, uncharged backbone modifications, morpholino modifications, 2′-O-methyl modifications, and phosphoramidate (see, e.g., Wagner (1995) NAT. MED. 1:1116-8). Thus, at least one 2′-hydroxyl group of the nucleotides on an RNA construct can be replaced by a chemical group, for example, by a 2′-amino or a 2′-methyl group.

Conjugating a ligand to an RNA construct can enhance its cellular absorption. In certain instances, a hydrophobic ligand is conjugated to the RNA construct to facilitate direct permeation of the cellular membrane. Alternatively, the ligand conjugated to the RNA construct is a substrate for receptor-mediated endocytosis. These approaches have been used to facilitate cell permeation of antisense oligonucleotides. For example, cholesterol has been conjugated to various antisense oligonucleotides resulting in compounds that are substantially more active compared to their non-conjugated analogs. See Manoharan et al., (2002) ANTISENSE & NUCLEIC ACID DRUG DEVELOPMENT 12(2):103-28. Other lipophilic compounds that have been conjugated to oligonucleotides include 1-pyrene butyric acid, 1,3-bis-O-(hexadecyl)glycerol, and menthol. One example of a ligand for receptor-mediated endocytosis is folic acid. Folic acid enters the cell by folate-receptor-mediated endocytosis. RNA constructs bearing folic acid would be efficiently transported into the cell via the folate-receptor-mediated endocytosis. Li and coworkers report that attachment of folic acid to the 3′-terminus of an oligonucleotide resulted in an 8-fold increase in cellular uptake of the oligonucleotide. Li, et al. (1998) PHARM. RES. 15:1540-5. Other ligands that have been conjugated to oligonucleotides include polyethylene glycols, carbohydrate clusters, cross-linking agents, porphyrin conjugates, and delivery peptides.

In certain instances, conjugation of a cationic ligand to oligonucleotides results in improved resistance to nucleases. Representative examples of cationic ligands are propylammonium and dimethylpropylammonium.

A composition that includes an RNA construct (it should be understood that the RNA construct compositions and administration methods referenced below include both RNA constructs and DNA vectors encoding an RNA construct) can be delivered to a subject by a variety of routes. Exemplary routes include intrathecal, parenchymal, intravenous, nasal, oral, parenteral and ocular delivery. An RNA construct can be incorporated into pharmaceutical compositions suitable for administration. For example, compositions can include one or more species of an RNA construct and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, intranasal, transdermal), oral or parenteral. Parenteral administration includes intravenous drip, subcutaneous, intraperitoneal or intramuscular injection, site-specific injection (e.g. delivery directly to a tumor or other site of disease), or intrathecal or intraventricular administration.

In general, an RNA construct can be administered by any suitable method. As used herein, topical delivery can refer to the direct application of an RNA construct to any surface of the body, including the eye, a mucous membrane, surfaces of a body cavity, or to any internal surface. Formulations for topical administration may include transdermal patches, ointments, lotions, creams, gels, drops, sprays, and liquids. Conventional pharmaceutical carriers, aqueous, powder or oily bases, thickeners and the like may be necessary or desirable. Topical administration can also be used as a means to selectively deliver the RNA construct to the epidermis or dermis of a subject, or to specific strata thereof, or to an underlying tissue.

Compositions for intrathecal or intraventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

An RNA construct can be administered to a subject by pulmonary delivery. Pulmonary delivery compositions can be delivered by inhalation by the patient of a dispersion so that the RNA construct within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, metered-dose devices, and dry powder dispersion devices. One of the benefits of using an atomizer or inhaler is that the potential for contamination is minimized because the devices are self-contained. Dry powder dispersion devices, for example, deliver drugs that may be readily formulated as dry powders. An RNA construct may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

An RNA construct can be modified such that it is capable of traversing the blood brain barrier. For example, the RNA construct can be conjugated to a molecule that enables the agent to traverse the barrier. Such modified RNA construct can be administered by any desired method, such as by intraventricular or intramuscular injection, or by pulmonary delivery, for example.

An RNA construct can be administered ocularly, such as to treat a retinal disorder, e.g., a retinopathy. For example, the pharmaceutical compositions can be applied to the surface of the eye or nearby tissue, e.g., the inside of the eyelid. They can be applied topically, e.g., by spraying, in drops, as an eyewash, or an ointment. Ointments or droppable liquids may be delivered by ocular delivery systems known in the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers. The pharmaceutical composition can also be administered to the interior of the eye, and can be introduced by a needle or other delivery device which can introduce it to a selected area or structure. The composition containing the RNA construct can also be applied via an ocular patch.

An RNA construct can be administered by an oral or nasal delivery. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

### EXAMPLES

### Example 1

### A Predicted Interaction Between miRNA and an mRNA Bound by SLBP was Used to Design an RNA Construct Capable of Binding an Endogenous Non-Coding RNA

To determine whether RNA constructs comprising a stem-loop could stably bind and inhibit non-coding RNAs, a search was performed for miRNAs that complex with mRNAs bound by SLBP (i.e., RNA molecules containing a stem-loop bound by SLBP). 600 miRNAs were found to be present in human chronic myelogenous lymphoblastoid K562 cell lysate using microfluidics miRNA array technology optimized to interrogate the comprehensive miRBase repertoire of identified miRNAs (LC Sciences, Houston, Tex.) Then, an RNA immunoprecipitation (RIP) targeting stem-loop binding protein (SLBP) was performed (Jain et al. (2011) supra; Jayaseelan et al. (2011) supra) and the results of that RIP were similarly analyzed. Approximately 60 miRNAs out of 600 miRNAs present in K562 lysate showed enrichment in the SLBP-RIP pool. In other words, approximately 60 miRNAs were found to complex with mRNAs bound by SLBP. Of these, 10 miRNA could not be accounted for using conventional miRNA targeting algorithms as predicted by informatic tools such as miRanda or PicTar. However, these 10 miRNA are predicted to bind through non-conventional three-way junction interactions as depicted in FIG. 3. Several of these mRNAs have flanks of the stem-loop that are almost perfectly complementary to the corresponding miRNA.

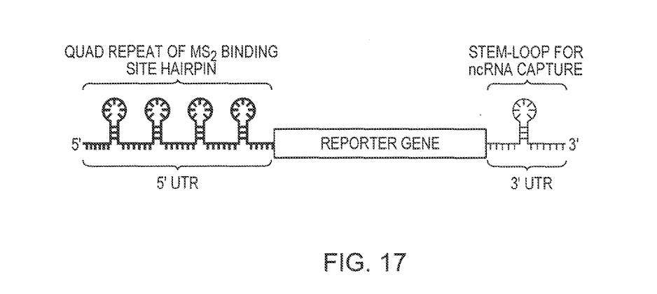

One of these 10 miRNA, miR-4298 (5′-CUGGGACAGGAGGAGGAGGCAG-3′ (SEQ ID NO:23)) and the portion of the 3′ UTR for HIST1H3c to which it binds (5′-CUGCCCGUUUCUUCCUCAUUGAAAAGGCUCUUUUCAGAGCCACUCA-3′ (SEQ ID NO:24)), was identified for further study. An RNA construct was created that has the naturally occurring 3′ UTR for HIST1H3c and a 5′ UTR that contained multiple copies of the MS2 Bacteriophage RBP RNA-target motif. This routinely used molecular tag is a small RNA stem-loop structure that is readily bound (typically as a duplex) by the bacteriophage MS2 coat-protein. The MS2-protein/RNA tagging combination has been widely used for capturing a targeted RNA and is naturally used by the virus to encapsidate its RNA genome. As depicted in FIG. 17, DNA templates were designed having a T7-promoter for RNA transcription followed by several MS2-RNA tags in tandem at the 5′-end of reporter construct. Downstream from this, the HSL-containing mRNA portion of the 3-way junction was introduced. T7-transcribed RNA was nucleofected into K562 cells and incubated for 4 hours. Cells were then lysed and miR-4298 was detected using SYBR®-Green based RT-PCR and compared to a control set of miRNAs to determine the relative concentrations of target and non-target miRNAs prior to imunoprecipitation. A standard RIP was then performed using ectopically added MS2-protein, and associated miRNA was detected by RT-PCR. As depicted in FIG. 18, miR-4298 was efficiently immunoprecipitated four control miRNAs, all of which were present at detectable levels (15-28 Ct) in the input material. One-way ANOVA was used for statistical analysis. A pValue=0.19 was observed with F-statistic being 4.9 and the F critical being 3.48 (meaning this was significant). This means that an ectopically introduced RNA construct is capable of binding to an endogenous non-coding RNA (e.g., a miRNA) in cells.

### Example 2

### Construction of an RNA Construct which Inhibited a miRNA

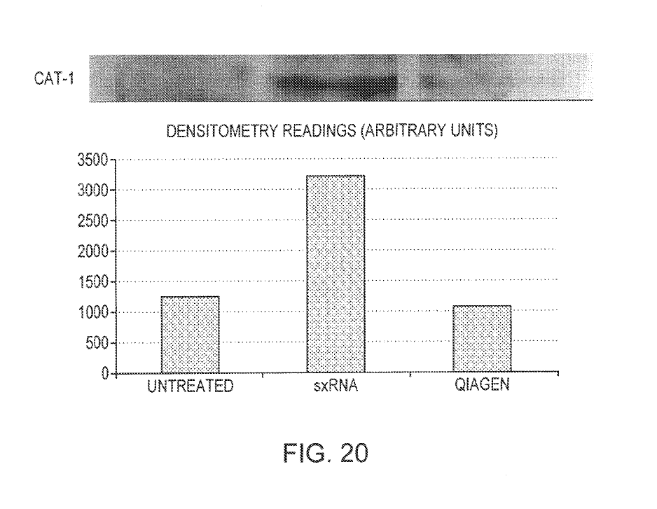

The RNA construct (i.e., sxRNA switch) shown in FIG. 19 (5′-CAAACACCAUUGUCAAAAGGCUCUUUUCAGAGCCACACUCCA-3′ (SEQ ID NO:25) was designed and assessed for its ability to inhibit miR-122 (5′-UGGAGUGUGACAAUGGUGUUUG-3′ (SEQ ID NO:26). The T7-transcribed sxRNA switch was transfected into Huh-7 cells (known to express high levels of miR-122) and the effect on Cationic Amino Acid Transporter-1 (CAT-1) expression, a confirmed miR-122 target (Bhattacharyya et al. (2006) “Relief of microRNA-Mediated Translational Repression in Human Cells Subjected to Stress,” CELL 125 (6):1111-1124) was determined Approximately 250,000 Huh-7 cells were transfected in duplicate with 1.5 μg (approximately 50 nM) (1) RNA construct (i.e., the sxRNA miR-122 anti-miR), (2) Qiagen miR-122 anti-miR or (3) no RNA, using the HiPerFect Transfection Reagent (Qiagen, Germantown, Md.). After 24 hrs. incubation, cells were lysed and the total protein was assessed using Western blot analysis. Total protein from each treatment was measured by Bradford analysis and 30 micrograms were loaded into each lane. After Western blotting with CAT-1 antibody (Santa Cruz Biotechnology, Santa Cruz, Calif.), CAT-1 quantity was determined using densitometric analysis and normalized to an unrelated protein. As can be seen in FIG. 20, CAT-1 expression increased nearly 3× in the presence of the sxRNA miR-122 anti-miR as compared to untreated cells or those treated with a competitor (Qiagen) miR-122 anti-miR, indicating that an RNA construct can be designed that inhibits the function of a non-coding RNA.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

### INCORPORATION BY REFERENCE

The entire disclosure of each of the patent documents, including U.S. Pat. No. 8,841,438, scientific publications, and other database references disclosed hereinabove is expressly incorporated herein by reference for all purposes.

What is claimed is:

1. A method for inhibiting a non-coding RNA, the method comprising:
contacting a non-coding RNA with an RNA construct, wherein the RNA construct comprises a non-naturally occurring, continuous sequence of ribonucleotide bases defining:
a stem-loop structure;
three way junction joining regions 3 ‘ and 5’ of the stem-loop structure;
a first region 5′ of the 5′ joining region comprising bases complementary to a 3′ region of the non-coding RNA;
a second region 3 ‘ of the 3’ joining region comprising bases complementary to a 5′ region of the non-coding RNA;
the base sequence of the first and second regions being selected to hybridize with complementary bases on the non-coding RNA, wherein
the non-coding RNA binds preferentially to the RNA construct as compared to a natural target.

2. The RNA construct of claim 1, wherein the base sequence of the first and second regions are spaced apart by an intermediate region on the non-coding RNA defining another three way junction joining region.

3. The method of claim 1, wherein the non-coding RNA is an miRNA, an siRNA, a piRNA, an snoRNA, or an lncRNA.

4. (canceled)

5. The method of claim 1, wherein the RNA construct comprises a modified nucleic acid base.

6. The method of claim 1, wherein the RNA construct further comprises a detectable marker.

7. The method of claim 6, wherein the detectable marker is a fluorescent moiety or biotin.

8. The method of claim 1, wherein the method further comprises transfecting a DNA encoding the RNA construct into a cell.

9-11. (canceled)

12. The method of claim 1, wherein the non-coding RNA is an miRNA and the RNA construct inhibits the function of the miRNA as determined by:
(a) measuring the quantity of a downstream target of the miRNA in the presence of the RNA construct;
(b) measuring the quantity of the downstream target of the miRNA in the absence of the RNA construct;
(c) determining that the quantity of the downstream target in the presence of the RNA construct is greater that the quantity of the downstream target the absence of the RNA construct.

13. The method of claim 12, wherein the downstream target is mRNA or protein.

14. The method of claim 1, wherein the reduction in free energy that occurs when the RNA construct binds the non-coding RNA is between about 1 kCal and about 10,000 kCal.

15. The method of claim 14, wherein the reduction in free energy that occurs when the RNA construct binds the non-coding RNA is between about 10 kCal and about 100 kCal.

16. The method of claim 1, wherein, the reduction in free energy that occurs when the non-coding RNA binds the RNA construct is selected from the group consisting of at least 1% more, at least 2% more, at least 5% more, at least 10% more, at least 25% more, at least 50% more at least 75% more, at least 95% more, at least 100% more, at least 200% more, and at least 500% more than the reduction in free energy that occurs when a non-coding RNA binds its natural target.

17. The method of claim 1, wherein the non-coding RNA is an miRNA, and at least 50% of the miRNA remains uncleaved by the RISC complex.

18. The method of claim 1, wherein
the RNA construct inhibits the non-coding RNA and
in the presence of the non-coding RNA, the construct assumes a stem-loop conformation promoting association with a cellular protein, whereby
association of the stem-loop with the cellular protein inhibits the cellular protein.

19. The method of claim 18, wherein the RNA construct inhibits the non-coding RNA by sequestering the non-coding RNA, and association of the stem-loop with the cellular protein inhibits the cellular protein by sequestering the cellular protein.

20. The method of claim 19, wherein inhibition of the non-coding RNA and cellular protein is lethal to the cell.

21-26. (canceled)

27. The method of claim 1, wherein the non-coding RNA is expressed preferentially in a cell type of a multicellular organism.

28. The method of claim 1, wherein the cell type is an infected cell or a neoplastic cell, and the non-coding RNA is expressed by an organism infecting the cell or by the neoplastic cell.

29. The method of claim 1, wherein the RNA construct comprises more than one said stem-loop structure.
